(19) United States
(12) Patent Application Publication
(10) Pub. No.: US 2026/0134971 A1
(43) Pub. Date: May 14, 2026

(54) METHOD FOR THE SELECTION OF DIET FOR A SUBJECT

(71) Applicant: FUNDACIÓ EURECAT, CERDANYOLA DEL VALLÈS (ES)

(72) Inventors: JOSEP MARIA DEL BAS PRIOR, TARRAGONA (ES); XAVIER ESCOTÉ MIRÓ, L'ESPLUGA DE FRANCOLÍ (ES); JUAN MARÍA ALCAIDE HIDALGO, TARRAGONA (ES); DAVID SUÑOL MORENO, BARCELONA (ES); MAR GALOFRE CARDO, BARCELONA (ES); BIOTZ GUTIÉRREZ ARECHEDERRA, CERDANYOLA DEL VALLÈS (ES); KATHERINE GIL CARDOSO, REUS (ES); ROGER MARINÉ CASADÓ, REUS (ES); ILARIA BONAVITA, CERDANYOLA DEL VALLÈS (ES); MIGUEL ANGEL RODRIGUEZ GOMEZ, REUS (ES); ANTONI CAIMARI PALOU, REUS (ES); NOEMI BOQUE TERRE, TARRAGONA (ES); VICENTE JORGE RIBAS RIPOLL, BARCELONA (ES)

(21) Appl. No.: 19/120,035

(22) PCT Filed: Oct. 10, 2023
(86) PCT No.: PCT/EP2023/078014

§ 371 Date: Apr. 10, 2025

(30) Foreign Application Priority Data
Oct. 11, 2022 (EP) 22382965.6

(51) Int. Cl. G16H 20/60; A23L 33/00

(52) U.S. Cl. CPC G16H 20/60; A23L 33/30

(57) ABSTRACT

The invention relates to a method for the selection of a diet for a subject based on health data, comprising determining in an isolated sample of the subject two or more markers of two or more metabolic pathway clusters (i.e., carbohydrate metabolism, lipid metabolism, inflammation, microbiota metabolism, oxidative stress); then a score is given to each cluster, and a diet is recommended considering the values of the scores. The invention also relates to a system for implementing this method.

This application claims the benefit of European Patent Application 22382965.6 filed Oct. 11, 2022.

### TECHNICAL FIELD

The present invention is framed in the field of nutrition and health science, to methods for the proper recommendation of diets to subjects, and to systems for carrying out the methods, in which several data of the subject are taken in consideration.

### BACKGROUND ART

Personalized nutrition (PN) has gained great attention in the last decade and is currently one of the top trends in nutritional sciences. This discipline offers the possibility of adapting eating behaviour to personal needs and preferences but mainly to harness optimal benefits. From the consumer's point of view, PN represents a natural path to empowerment, facilitating decision-taking processes impacting on very different domains of their lives, such as physical activity performance, mental and physical wellbeing, or overall health improvement. Moreover, PN is also gaining relevance from the perspective of healthcare systems, since a wide array of highly prevalent non-communicable diseases that put a high burden on the system, are directly linked with dietary patterns and eating behaviour. A well-known example is obesity and obesity-related diseases, which are tightly linked to unbalanced behaviours in terms of physical activity, mental distress, and inappropriate eating patterns. Unfortunately, it has been repeatedly demonstrated that general nutrition recommendations, even those adopted by national and international polices, are not sufficient to promote the required changes in eating behaviours of individuals. Likely PN will be most successful if it specifically addresses metabolic deviations that can be nutritionally targeted by specific dietary choices, with the outcomes measured and made visible to the consumer. In this process, specific genetically determined susceptibilities and risks may be considered.

The establishment of personal metabolic profiles, of metabotyping individuals, allows classification of individuals according to their metabolic signature, which in turn can be associated to health status, dietary patterns, and response to interventions. Nevertheless, although metabolic markers can be currently applied to obtain a precise overview of the state of the metabolism of the person at the metabolite level, nutritional recommendations are still far from the precision that is needed to fulfil the specific requirements of everyone. This apparent gap is a consequence of the lack of knowledge on the effects of individual eating behaviours on specific elements of metabolism, since the complexity of food itself is further confounded by the complex processes of digestion, absorption, and metabolism, and interactions and signalling properties of food components, which in turn are dependent on individual intrinsic and environmental factors

The European Patent EP3529379B1 discloses a system and a method for implementing meal selection based on vitals, genotype, and phenotype of a subject, in which the metabolic adaptability of a subject is determined through the analysis of the user's blood following consumption of a multi-nutrient challenge beverage, wherein the multi-nutrient challenge beverage includes a) from 44 to 57 grams total fats; b) 75±15 grams total carbohydrates; and c) 20±3 grams total protein. Insulin, glucose, and triglycerides are determined in a blood sample. The authors illustrate that by means of this system and method, in combination with the eating preferences of the subject, a proper selection of meal ingredients can be sown. However, these method and system imply the need of the previous consumption of a beverage, which makes them complex to implement.

All in all, there remains a further need for further methods for the nutritional recommendations to reflect comprehensively a person's individuality and goals.

### SUMMARY OF INVENTION

Inventors have determined an advantageous mode of selecting a subject for a predetermined diet, or in other words, a way to recommend a particular diet for a subject from the analysis of certain markers, including metabolic informative markers and optionally other features of the subject, such as particular genotypes or physical parameters (i.e., body-mass index, race, gender, age, etc.).

The method for such a selection is of special interest for healthy subjects that want to follow or to know the most appropriate diet according to their features. Thus, although the method of selection is useful in case of certain diseases, it also allows for maintaining or likely maintaining or preserving a healthy status in case of no-disease or declared disease.

As will be seen in the examples, the method of the invention allowed the selection of an appropriate diet that finally led to the modulation of the markers indicating the diet to be selected in benefit of the subject. Therefore, the method is directly applicable to the selection of an effective diet.

This method goes further to the routine control analytics currently taken to determine if certain organs or systems are within the established as normal operation or functioning, and which method also allow the expert to administer a medical regimen and/or a diet recommendation. The method of the invention goes some steps forward, in terms that certain markers of different informative clusters are determined in an isolated sample, and then the presence, levels or absence of them are given a value that is considered for taking a decision in conjunction with the values of the markers obtained for the other clusters. Thus, the decision is taken considering the values in relation with all the analysed metabolic informative clusters.

According to the inventor's knowledge, this is the first time the proposed groups of markers of certain clusters are taken together for the indicated purpose. It is also the first time several markers of a cluster are computed together to give relevant information regarding the optimal diet for a subject.

Thus, a first aspect of the invention is a method for the selection of a diet for a subject based on health data, comprising:
(i) determining in an isolated sample of the subject two or more markers of two or more of the following metabolic clusters:
(a) carbohydrate metabolism;
(b) lipid metabolism;
(c) inflammation;
(d) microbiota metabolism; and
(e) oxidative stress;
(ii) giving a score to each cluster, which score results from a function considering the levels and/or concentrations of the two or more markers in the sample;
(iii) comparing the scores obtained for the clusters (a) to (e) to determine a gradation between them; and
(iv) recommending a diet for the subject considering the said gradation and optionally the individual score value in relation to a reference value for that score.

Therefore, for example after the performance of the method of the first aspect, a diet that fits with the metabolic cluster which has the highest score is recommended to the subject. This selection, as will be seen in the examples will reduce the score of said cluster within a period of time.

If the highest score is that of the carbohydrate metabolism, this will mean that the metabolic pathways of carbohydrate metabolism are the most likely to trigger or cause a nutritional disorders or diseases associated to metabolic disorders.

With the scoring of all the analysed clusters, a diet is recommended to adjust at least one of the scores of the said clusters.

In another example, if one of the analysed clusters gives a score out of a reference interval or above or below a cut-off value for that score, a diet is recommended to adjust said score to the reference or to approach its cut-off value.

The skilled person will understand that the essence of the invention lies in the combination of the different biomarkers (i.e., at least two), defining a metabolic signature, in which each of the markers is associated with a slightly different aspect of the same signature. In other words, the essence lies in considering or transforming the different biomarkers as a single composite biomarker (i.e., mathematically translated to a score for a particular cluster).

Therefore, the method proposes a procedure to determine a personalized nutrition recommendation based on information obtained from metabolomic clusters, and in some embodiments each (bio) markers in a cluster are weighted according to their relative significance within the cluster.

The rationale behind the effectivity of the method is, without being bound to any theory, that at least in part dysregulation of the overarching processes (i.e., those global and complex processes that control health status) manifest as measurable alterations of different elements of metabolism under a fixed condition, such as an overnight fast. The metabolic elements that likely reflect the five overarching processes are lipid metabolism, carbohydrate metabolism, systemic inflammation (i.e., inflammation), oxidative stress and microbiome status (i.e., microbiota metabolism). Because sustained alterations of these five elements are associated with the likely onset of different diseases, they are also referred in this description as “core health processes”.

The main characteristic of core health processes is that each one recapitulates different and complementary aspects of metabolism and can be conceived as independent clusters of different metabolites and proteins that are currently recognized as established clinical biomarkers or in advanced research stages (i.e., meta-analyses of clinical studies) as indicators of specific conditions or metabolic alterations. These emerging and consolidated biomarkers, combined by algorithms assisted by machine learning techniques, surprisingly provided a measurement of the state of each core health process. The main advantage of this approach is that each core health process is composed by a combination of different biomarkers (i.e., at least two), which comprise a health signature, each of the markers associated with a slightly different aspect of the same signature. Thus, in terms of physiology and health-to-disease progression, the information provided by these biomarkers is complementary. For example, combining these biomarkers by taking into consideration both the relevance of health-to-disease progression and blood concentrations, resulted in a more sensitive manner of capturing changes in inflammation homeostasis. Therefore, the inventors propose that subtle undetectable changes in a metabolic process when biomarkers are considered separately, become detectable if different biomarkers are considered as a single composite biomarker. At the same time, important changes in a single but relevant biomarker of the signature do not affect the capacity of detection despite the unlikely situation of other biomarkers remaining unaltered.

As indicated, for the carrying out of the method, a machine learning and/or particularly developed systems allow for a best implementation of the same.

A second aspect of the invention is, thus, a system for the selection of a diet for a subject based on health data, comprising:
a database that stores data determined in an isolated sample of the subject of the two or more markers of two or more of the following metabolic clusters:
(a) carbohydrate metabolism;
(b) lipid metabolism;
(c) inflammation;
(d) microbiota metabolism; and
(e) oxidative stress;
   a memory that stores program instructions, including program instructions that are capable of implementing: (i) a function to get a score of the metabolic clusters from the levels and/or concentrations of the two or more markers in the sample; (ii) a comparison of the scores to determine a gradation between them; and (iii) a diet selection for the subject based on the score of the metabolic clusters and their comparison; and
   a processor, coupled to the database and the memory, that when executing the program instructions causes the function to get a score of the metabolic clusters from the levels and/or concentrations of the two or more markers in the sample, causes the comparison of the scores to determine a gradation between them, and causes a diet selection for the subject based on the score of the metabolic clusters and their comparison.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As used herein, the indefinite articles “a” and “an” are synonymous with “at least one” or “one or more.” Unless indicated otherwise, definite articles used herein, such as “the” also include the plural of the noun.

For the purposes of the present invention, any ranges given include both the lower and the upper endpoints of the range.

The term “marker” according to this description includes any of the levels of substances (i.e., metabolites, carbohydrates, lipids, proteins, peptides, mRNA, miRNA, etc.), in isolated samples or the presence of certain mutations, or genotypes, such as single nucleotide polymorphisms (SNPs), or other conditions, such as certain diseases.

The expression “carbohydrate metabolism” relates to the whole of the biochemical processes responsible for the metabolic formation, breakdown, and interconversion of carbohydrates in living organisms. The metabolic pathways included under the carbohydrate metabolism umbrella are the glycolysis, the gluconeogenesis, the glycogenesis, the pentose phosphate pathway, the fructose metabolism, and the galactose metabolism. Metabolic control elements of such metabolic pathways such as insulin, adiponectin, leptin, HOMA-IR, certain amino acids, among other, are also included under the carbohydrate metabolism umbrella. The skilled person in the art will identify all these pathways and the enzymes, receptors and compounds involved therein. Thus, the expression “cluster of carbohydrate metabolism” or “carbohydrate metabolism cluster” used in this description relates to the group of markers known to give information about the status quo in which all the biochemical and cell events involved in the carbohydrate metabolism are at a particular moment of the life of the subject (i.e., glucose, glutamate, insulin, leptin, etc.).

The expression “lipid metabolism” relates to the synthesis and degradation of lipids in cells, involving the breakdown or storage of fats for energy and the synthesis of structural and functional lipids, such as those involved in the construction of cell membranes. It includes the biochemical pathways known as “lipid digestion”, lipid absorption, lipid transportation, lipid storage, lipid catabolism and lipid biosynthesis. The skilled person in the art will identify all these pathways and the enzymes, receptors and compounds involved therein. The expression “cluster of lipid metabolism” or “lipid metabolism cluster” used in this description relates to the group of markers known to give information about the status quo in which all the biochemical and cell events involved in the lipid metabolism are at a particular moment of the life of the subject (i.e., Low Density Lipoprotein (LDL), High Density Lipoprotein (HDL), triglycerides, choline, etc.).

The term “inflammation” relates to the complex biological response of body tissues to harmful stimuli, such as pathogens or particles thereof, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair. The expression “inflammation cluster” used in this description relates to the group of markers known to give information about the status quo or picture of the biochemical and cell events involved in the inflammatory processes are at a particular moment of the life of the subject (i.e., levels of C-reactive protein (CRP), Monocyte chemoattractant protein-1 (MCP-1), etc.).

“Microbiota metabolism” relates to the many microbe-derived small molecules that are present due to the gut microbiota (GM) of a subject. The symbiotic relation between GM and host produces a myriad of metabolic signatures, and the technological advances in GM metabolomics are progressively decoding the host-microbes metabolic interaction. Thus, the “microbiota metabolism cluster” according to this description relates to the group of possible markers in an isolated sample that will give information about the type and status of the microbiota (i.e., succinate, lactate, TMA, etc.). For a more detailed comprehension of the term see Vernocchi, P., Del Chierico, F., & Putignani, L. (2020). Gut Microbiota Metabolism and Interaction with Food Components. International journal of molecular sciences, 21 (10), 3688. https://doi.org/10.3390/ijms21103688.

Under the expression “oxidative stress” the skilled person will understand as the situation that reflects an imbalance between the systemic manifestation of reactive oxygen species (ROS) and a biological system's ability to readily detoxify the reactive intermediates or to repair the resulting damage. Disturbances in the normal redox state of cells can cause toxic effects through the production of peroxides and free radicals that damage all components of the cell, including proteins, lipids, and DNA. A “cluster of oxidative stress” or “oxidative stress cluster” is relates to the group of markers known to give information about the presence ad degree of this imbalance at a particular moment of the life of the subject (i.e., levels of 8-iso-prostaglandin F2α (8-iso-PGF2α), uric acid, dimethylglycine, etc.).

According to this description, the term “subject” encompasses an animal, more in particular a mammal and even more in particular a human.

As previously indicated, a first aspect of the invention relates to a method for the selection of a diet for a subject based on health data, comprising:
(i) determining in an isolated sample of the subject two or more markers of two or more of the following metabolic clusters:
(a) carbohydrate metabolism;
(b) lipid metabolism;
(c) inflammation;
(d) microbiota metabolism; and
(e) oxidative stress;
(ii) giving a score to each cluster, which score results from a function considering the levels and/or concentrations of the two or more markers in the sample;
(iii) comparing the scores obtained for the two or more clusters (a) to (e) to determine a gradation between them; and
(iv) recommending a diet for the subject considering the said gradation and optionally the individual score value in relation to a reference value for that score.

In a particular embodiment of the method of the first aspect, step (iii) comprises comparing the scores obtained for two or more of the clusters (a) to (e) to determine the highest score, and step (iv) recommending a diet for the subject considering the said higher of the scores for the two or more of the clusters (a) to (e).

In another particular embodiment of the first aspect, the function to get the score of each of the analysed clusters in step (ii) comprises giving a value to each of the markers of the cluster, which value results from the levels and/or concentration of the marker detected in the isolated sample modified by a factor (or weight).

In a particular embodiment the factor is a rational number from −1 to +1, being the absolute value from/0/ to /1/ and resulting from the relative significance of the marker within the whole set of markers in that cluster. All this means that the levels and/or concentration of each marker are multiplied by a value (factor or weight) according to the significance of that marker in the cluster. The “significance” of a marker within a group of markers is thus finally given a numerical value (rational), determined for example as indicated in the following paragraph. Other methods could be used to give such a numerical value that will be known by the skilled person in the art, such as regression or variable selection methods.

The calculation of the absolute value of the rationales from −1 to +1 for each of the analyzed markers in a determined cluster may be done for example by giving a relative punctuation (score individual marker (SIM)) to each of the markers playing in that cluster in function of the significance in the said cluster, said significance decided according to the observations of the inventors, common general knowledge about that marker in the particular metabolic pathways (i.e., from the scientific evidence) as well as with machine learning (ML) methods (e.g. PLS Regression). The punctuation is thus a quantitative measure of the significance of the marker in that group of markers conforming a cluster. For example, a punctuation of 25 is given by the inventors to LDL. The remaining markers in the lipid metabolism are assigned a punctuation in the same way and under the same criteria. Thus, the SIM value itself is an arbitrarily assigned number to a marker, the important point here is that the relation between SIM values of different biomarkers has to be consistent (i.e. proportional) to the respective significances of each marker within the cluster.

In a particular embodiment, the SIM values for each of the two or more markers determined for the selected clusters are proportionally related to the SIM values for each other determined markers within the same cluster in a proportion falling within the ranges given in Table F, Table G, Table H, Table I and Table J, wherein X is selected from 0.75 to 0. In a more particular embodiment X is selected from 0.5 to 0. In a more particular embodiment X is selected from 0.45 to 0, 0.40 to 0 or 0.35 to 0. In a more particular embodiment X is selected from 0.30 to 0. In a more particular embodiment X is selected from 0.25 to 0, 0.20 to 0 or 0.15 to 0. In a more particular embodiment X is selected from 0.10 to 0 or 0.05 to 0. In a more particular embodiment X is selected from 0.75, 0.70, 0.65, 0.60, 0.55, 0.50, 0.45, 0.40, 0.35, 0.30, 0.25, 0.20, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0. In a more particular embodiment X is selected from 0.50, 0.45, 0.40, 0.35, 0.30, 0.25, 0.20, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0. In a more particular embodiment X is selected from 0.30, 0.25, 0.20, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0. In a more particular embodiment X is selected from 0.10, 0.09, 0.08, 0.07, 0.06, 0.05, 0.04, 0.03, 0.02, 0.01, or 0. In a more particular embodiment X is 0.

In another particular embodiment, the SIM values for each of the two or more markers determined for the selected clusters are ordered within each cluster according to the order of tables A-E.

Once a punctuation has been assigned to the markers playing in that cluster, then all the punctuations are added and each of them are divided by the total punctuation to obtain the weight or factor of the marker within that cluster. For example, if the total punctuation of the markers considered for the lipid metabolism cluster amount to 182 in the previous example where LDL has a punctuation of 25 (i.e., considering all other markers not listed now for simplification), the weight (in percentage) of LDL in that cluster would be 13.7% [(25/182)*100], or which is the same the factor that will multiply the levels of LDL determined in the isolated sample will have an absolute value of 0.137. The rational will then be a positive or a negative rational, for example by giving a negative value to the markers that their levels and/or concentration decrease in the assayed sample in case of, or at risk of, a disease related with that metabolic cluster (referred as “Reverse” in Tables); and by giving a positive value to the markers that their levels and/or concentration increase in the assayed sample in case of, or at risk of, a disease related with that metabolic cluster (referred as “Normal” in Tables). For example, the rational from −1 to +1 that would correspond for multiplying the determined levels of LDL in the previous example would be (+0.137), since it is known that LDL levels are increased over in case of a disease related with lipid metabolism (e.g., obesity, atherosclerosis, etc.).

In a particular embodiment of the first aspect, the weight or factor of a particular marker within a cluster, which provides the measure of the significance of the marker in the cluster, is calculated according to the formula (1):

$${weight ( cluster )}_{n} = \frac{{SIM ( cluster )}_{n}}{{\sum }_{n = 1}^{Ncluster} {SIM ( cluster )}_{n}} \qquad ( 1 )$$

wherein
   “weight (cluster)” is the weight or factor selected from a rational from −1 to +1;
   “SIM (cluster)” is the Score of individual marker (punctuation) of a particular marker determined in the isolated sample for a particular cluster;
   “n” is one specific biomarker of the cluster; and
   “Ncluster” is the total amount of biomarkers for that specific cluster.

In a more particular embodiment, the SIM for each marker is as disclosed in the SIM (P) (Score of individual marker (punctuation) columns of Tables A-E. In a more particular embodiment, the SIM for each marker is as disclosed in the SIM (P) (Score of individual marker (punctuation)) columns of Tables 1-5.

In some embodiments, the specific punctuations for each marker, and so the corresponding deduced factors within a group of markers for that cluster can be tuned in function of the analysed population. Thus, they can be customized considering several aspects of the analysed cohorts, such as the race, geographical features, particular disease, etc. Or they can be punctuated in function of the different analysed markers relatively considered each one with the others.

As previously announced, the essence of the present invention lies in the combination of the different biomarkers (i.e., at least two), which comprise or define a health signature, each of the markers associated with a slightly different aspect of the metabolism, but indicative of the same signature. In other words, the essence lies in considering or transforming the different biomarkers (i.e., levels of individualized markers in the sample) as a single composite biomarker (i.e., mathematically translated to a score for a particular cluster).

Thus, the skilled person will understand that the essence does not lie in these specific rational numbers (i.e., from −1 to +1, or weight in percentage) that will ultimately be used to multiply the levels of the markers in the function to obtain a score for that cluster.

In another particular embodiment of the method, optionally in combination with the embodiments above or below, the function to get the score of each cluster in step (ii) comprises a linear combination of the two or more markers of the cluster according to the following formula (2):

$$Score ( cluster ) = {\sum }_{n = 1}^{Ncluster} ( {weight ( cluster )}_{n} * {biomarker ( cluster )}_{n} ) \qquad ( 2 )$$

wherein
   “biomarker (cluster)” is the level and/or concentration of a particular marker determined in the isolated sample for a particular cluster;
   “weight (cluster)” is the weight or factor selected from a rational from −1 to +1;
   “n” is one specific biomarker of the cluster; and
   “Ncluster” is the total amount of biomarkers for that specific cluster.

In a particular embodiment, the “weight (cluster) n” in the formula (2) is calculated as described above, in particular, by using the formula (1).

In a more particular embodiment, the value given to each of the markers (i.e., “biomarker (cluster) n” in the formula (2) above) is an amount calculated as a Z-score value, wherein the Z-score for a marker is calculated by subtracting the reference population mean from the level and/or concentration and then dividing the difference by the standard deviation of the reference population.

Thus, in a particular embodiment the biomarker (cluster), can be referred as ZScore biomarker (cluster)ₙ, and calculated by using the formula (3):

$$ZScore {biomarker ( cluster )}_{n} = \frac{{biomarker ( cluster )}_{n} - {biomarker ( reference population )}_{n}}{SD {biomarker ( reference population )}_{n}} \qquad ( 3 )$$

wherein
   “ZScore biomarker (cluster)” is the level and/or concentration of a particular marker determined in the isolated sample for a particular cluster calculated as a Z-score;
   “biomarker (reference population)” is the level and/or concentration of a particular marker determined in the reference population;
   “biomarker (cluster)” is the level and/or concentration of a particular marker determined in the isolated sample for a particular cluster;
   “SD biomarker (reference population)” is the Standard Deviation (SD) of a particular marker determined in reference population; and
   “n” is one specific biomarker of the cluster.

The skilled person will recognize the suitable reference population. For example, if the test is performed with a subject within the European territory, a population representative of general European population is the most appropriate to be used to obtain such a Z-score that will be computed in the function of to obtain the score of the cluster.

In a particular embodiment, the method for the selection of a diet for a subject based on health data, comprises:
(i) determining in an isolated sample of the subject two or more markers of two or more of the following metabolic clusters:
(a) carbohydrate metabolism;
(b) lipid metabolism;
(c) inflammation;
(d) microbiota metabolism; and
(e) oxidative stress;
wherein:
   the markers of the (a) carbohydrate metabolism cluster are selected from glucose, homeostatic model assessment of insulin resistance (HOMA-IR), glutamate, uric acid, leptin, adiponectin, insulin, tyrosine, propionylcarnitine, lactate, valine, leucine, isoleucine, phenylalanine, and glutamine;
   the markers of the (b) lipid metabolism cluster are selected from LDL-cholesterol, total cholesterol, total polyunsaturated fatty acids (PUFAs (total), HDL-cholesterol, saturated fatty acids, triglycerides, total monounsaturated fatty acids (MUFAs (total)), total lysophosphatidylcholine (LPC total), linoleic acid, docosahexaenoic acid (DHA), oleic acid, choline, 3-hydroxybutyrate, propionylcarnitine, adiponectin, and leptin;
   the markers of the (c) inflammation cluster are selected from C-reactive protein (CRP), N-acetylglycoproteins, Monocyte chemoattractant protein-1 (MCP-1), tumour necrosis factor α (TNFα), interleukin-6 (IL-6), interleukin-10 (IL-10), saturated fatty-acids (SFAs), Soluble intercellular adhesion molecule-1 (sICAM1), total Lysophosphatidylcholine (LPC total), lipopolysaccharide binding protein (LBP), docosahexaenoic acid (DHA), soluble cluster of differentiation 14 (sCD14), linoleic acid, and total polyunsaturated fatty acids (PUFAs (total);
   the markers of the (d) microbiota metabolism cluster are selected from trimethylamine (TMA) N-oxide of trimethylamine (TMAO), betaine, choline, dimethylamine (DMA), dimethylglycine, lipopolysaccharide binding protein (LBP), succinate, lactate, and acetate; and
   the markers of the (e) oxidative stress cluster are selected from 8-iso-prostaglandin F2α (8-iso-PGF2α), 8-Hydroxyguanosine (8-OHdG), oxidized low density lipoprotein (LDLox), uric acid, allantoin, betaine, pseudouridine, dimethylglycine, methionine, and glycine;
(ii) giving a score to each cluster, which score is calculated according to the following formula (2):

$$Score ( cluster ) = {\sum }_{n = 1}^{Ncluster} ( {weight ( cluster )}_{n} * {biomarker ( cluster )}_{n} ) \qquad ( 2 )$$

wherein
   “biomarker (cluster)” is the level and/or concentration of a particular marker determined in the isolated sample for a particular cluster, the concentration optionally calculated as a Z-score;
   “n” is one specific biomarker of the cluster;
   “Ncluster” is the total amount of biomarkers for that specific cluster; and
   “weight (cluster)” is the weight or factor selected from a rational from −1 to +1, which is calculated according to the following formula (1):

$${weight ( cluster )}_{n} = \frac{{SIM ( cluster )}_{n}}{{\sum }_{n = 1}^{Ncluster} {SIM ( cluster )}_{n}} \qquad ( 1 )$$

wherein
   “n” and “Ncluster” are as defined above; and
   “SIM (cluster)” is the Score of individual marker (punctuation) of a particular marker determined in the isolated sample for a particular cluster; wherein the SIM value for each marker is proportionally related to the SIM values for the other determined markers within the same cluster in a proportion falling within the ranges given in Tables F-J, wherein X is selected from 0.50 to 0;
(iii) comparing the scores obtained for the two or more of clusters (a) to (e) to determine a gradation between them; and
(iv) recommending a diet for the subject considering the said gradation and optionally the individual score value in relation to a reference value for that score.

In a particular embodiment of the previous embodiment, X is selected from 0.30 to 0. In a more particular embodiment X is selected from 0.20 to 0. In a more particular embodiment X is selected from 0.10 to 0. In a more particular embodiment X is 0.

In another particular embodiment of the first aspect, the two or more markers of the (a) carbohydrate metabolism cluster are selected from glucose, homeostatic model assessment of insulin resistance (HOMA-IR), glutamate, uric acid, leptin, adiponectin, insulin, tyrosine, propionylcarnitine, lactate, valine, leucine, isoleucine, phenylalanine, and glutamine.

In another particular embodiment of the first aspect, the two or more markers of the (a) carbohydrate metabolism cluster are selected from glucose, homeostatic model assessment of insulin resistance (HOMA-IR), glutamate, leptin, adiponectin, valine, leucine, and isoleucine.

In also another particular embodiment of the method of the first aspect, the two or more markers of the (b) lipid metabolism cluster are selected from LDL-cholesterol, total cholesterol, total polyunsaturated fatty acids (PUFAs (total)), HDL-cholesterol, saturated fatty acids, triglycerides, total monounsaturated fatty acids (MUFAs (total)), total lysophosphatidylcholine (LPC total), linoleic acid, docosahexaenoic acid (DHA), oleic acid, choline, 3-hydroxybutyrate, propionylcarnitine, adiponectin, and leptin.

In another particular embodiment of the method of the first aspect, the two or more markers of the (b) lipid metabolism cluster are selected from LDL-cholesterol, total cholesterol, total polyunsaturated fatty acids (PUFAs (total)), HDL-cholesterol, saturated fatty acids, triglycerides, and total monounsaturated fatty acids (MUFAs (total)).

In also another particular embodiment of the method of the first aspect, the two or more markers of the (c) inflammation cluster are selected from C-reactive protein (CRP), N-acetylglycoproteins, Monocyte chemoattractant protein-1 (MCP-1), tumor necrosis factor α (TNFα), interleukin-6 (IL-6), interleukin-10 (IL-10), saturated fatty-acids (SFAs), Soluble intercellular adhesion molecule-1 (sICAM1), total Lysophosphatidylcholine (LPC total), lipopolysaccharide binding protein (LBP), docosahexaenoic acid (DHA), soluble cluster of differentiation 14 (sCD14), linoleic acid, and total polyunsaturated fatty acids (PUFAs (total).

In also another particular embodiment of the method of the first aspect, the two or more markers of the (c) inflammation cluster are selected from C-reactive protein (CRP), N-acetylglycoproteins, Monocyte chemoattractant protein-1 (MCP-1), tumour necrosis factor α (TNFα), interleukin-6 (IL-6), and interleukin-10 (IL-10).

In also another particular embodiment of the method of the first aspect, the two or more markers of the (d) microbiota metabolism cluster are selected from trimethylamine (TMA), N-oxide of trimethylamine (TMAO), betaine, choline, dimethylamine (DMA), dimethylglycine, lipopolysaccharide binding protein (LBP), succinate, lactate, and acetate.

In also another particular embodiment of the method of the first aspect, the two or more markers of the (d) microbiota metabolism cluster are selected from trimethylamine (TMA) N-oxide of trimethylamine (TMAO), betaine, and choline.

In also another particular embodiment of the method of the first aspect, the two or more markers of the (e) oxidative stress cluster are selected from 8-iso-prostaglandin F2α (8-iso-PGF2α), 8-Hydroxyguanosine (8-OHdG), oxidized low density lipoprotein (LDLox), uric acid, allantoin, betaine, pseudouridine, dimethylglycine, methionine and glycine.

In also another particular embodiment of the method of the first aspect, the two or more markers of the (e) oxidative stress cluster are selected from 8-iso-prostaglandin F2α (8-iso-PGF2α), 8-Hydroxyguanosine (8-OHdG), and oxidized low density lipoprotein (LDLox).

In a more particular embodiment of the first aspect, in step (i) the following markers of the (a) cluster are determined in the isolated sample: glucose, homeostatic model assessment of insulin resistance (HOMA-IR), glutamate, uric acid, leptin, adiponectin, insulin, tyrosine, propionylcarnitine, lactate, valine, leucine, isoleucine, phenylalanine, and glutamine.

In also another more particular embodiment of the first aspect, in step (i) the following markers of the (b) cluster are determined in the isolated sample: LDL-cholesterol, total cholesterol, total polyunsaturated fatty acids (PUFAs (total)), HDL-cholesterol, saturated fatty acids, triglycerides, total monounsaturated fatty acids (MUFAs (total)), total Lysophosphatidylcholine (LPC total), linoleic acid, docosahexaenoic acid (DHA), oleic acid, choline, 3-hydroxybutyrate, propionylcarnitine, adiponectin, and leptin.

In also another more particular embodiment of the first aspect, in step (i) the following markers of the (c) cluster are determined in the isolated sample: C-reactive protein (CRP), N-acetylglycoproteins, Monocyte chemoattractant protein-1 (MCP-1), tumor necrosis factor α (TNFα), interleukin-6 (IL-6), interleukin-10 (IL-10), saturated fatty-acids (SFAs), Soluble intercellular adhesion molecule-1 (sICAM1), total Lysophosphatidylcholine (LPC total), lipopolysaccharide binding protein (LBP), docosahexaenoic acid (DHA), soluble cluster of differentiation 14 (sCD14), linoleic acid, and total polyunsaturated fatty acids (PUFAs (total).

In also another more particular embodiment of the first aspect, in step (i) the following markers of the (d) cluster are determined in the isolated sample: trimethylamine (TMA) N-oxide of trimethylamine (TMAO), betaine, choline, dimethylamine (DMA), dimethylglycine, lipopolysaccharide binding protein (LBP), succinate, lactate, and acetate.

In also another more particular embodiment of the first aspect, in step (i) the following markers of the (e) cluster are determined in the isolated sample: 8-iso-prostaglandin F2α (8-iso-PGF2α), 8-Hydroxyguanosine (8-OHdG), oxidized low density lipoprotein (LDLox), uric acid, allantoin, betaine, pseudouridine, dimethylglycine, methionine and glycine.

The markers contemplated in the present invention may be determined according to methods which are well known and available to the skilled person.

In another particular embodiment of the method of the first aspect, it comprises in step (i) determining in the isolated sample two or more markers of one, two, three, four or the five of the following metabolic clusters:
(a) carbohydrate metabolism;
(b) lipid metabolism;
(c) inflammation;
(d) microbiota metabolism; and
(e) oxidative stress.

In another particular embodiment two or more markers of (a) and two or more markers of (b); two or more markers of (a) and two or more markers of (c); two or more markers of (a) and two or more markers of (d); two or more markers of (a) and two or more markers of (e); two or more markers of (b) and two or more markers of (c); two or more markers of (b) and two or more markers of (d); two or more markers of (b) and two or more markers of (e); two or more markers of (c) and two or more markers of (d); two or more markers of (c) and two or more markers of (e); and two or more markers of (d) and two or more markers of (e).

In another particular embodiment, two or more markers of each of the three of the clusters (a), (b) and (c); (a), (b) and (d); (a), (b) and (e); (b), (c) and (d); (b), (c) and (e); (c), (d) and (e); and (a), (c) and (e).

In another particular embodiment, two or more markers of (a), two or more markers of (b), two or more markers of (c) and two or more markers of (d); two or more markers of (a), two or more markers of (b), two or more markers of (c) and two or more markers of (e);
two or more markers of (b), two or more markers of (c), two or more markers of (d) and two or more markers of (e); two or more markers of (a), two or more markers of (c), two or more markers of (d) and two or more markers of (e); and two or more markers of (a), two or more markers of (b), two or more markers of (d) and two or more markers of (e).

In yet another more particular embodiment of the method according to the first aspect, in step (i) the following markers of the (a) cluster are determined: glucose, homeostatic model assessment of insulin resistance (HOMA-IR), glutamate, uric acid, leptin, adiponectin, insulin, tyrosine, propionylcarnitine, lactate, valine, leucine, isoleucine, phenylalanine, and glutamine;
the following markers of the (b) cluster are determined in the isolated sample: LDL-cholesterol, total cholesterol, total polyunsaturated fatty acids (PUFAs (total), HDL-cholesterol, saturated fatty acids, triglycerides, total monounsaturated fatty acids (MUFAs (total)), total Lysophosphatidylcholine (LPC total), linoleic acid, docosahexaenoic acid (DHA), oleic acid, choline, 3-hydroxybutyrate, propionylcarnitine, adiponectin, and leptin;
the following markers of the (c) cluster are determined in the isolated sample: C-reactive protein (CRP), N-acetylglycoproteins, Monocyte chemoattractant protein-1 (MCP-1), tumor necrosis factor α (TNFα), interleukin-6 (IL-6), interleukin-10 (IL-10), saturated fatty-acids (SFAs), Soluble intercellular adhesion molecule-1 (sICAM1), total Lysophosphatidylcholine (LPC total), lipopolysaccharide binding protein (LBP), docosahexaenoic acid (DHA), soluble cluster of differentiation 14 (sCD14), linoleic acid, and total polyunsaturated fatty acids (PUFAs (total);
the following markers of the (d) cluster are determined in the isolated sample: trimethylamine (TMA) N-oxide of trimethylamine (TMAO), betaine, choline, dimethylamine (DMA), dimethylglycine, lipopolysaccharide binding protein (LBP), succinate, lactate, and acetate; and
the following markers of the (e) cluster are determined in the isolated sample: 8-iso-prostaglandin F2α (8-iso-PGF2α), 8-Hydroxyguanosine (8-OHdG), oxidized low density lipoprotein (LDLox), uric acid, allantoin, betaine, pseudouridine, dimethylglycine, methionine and glycine.

In also another particular embodiment of the method of the first aspect, it further comprises determining one or more clinical or subject parameters selected from the group consisting of sex, height, weight, race, blood pressure, waist circumference, body mass index (BMI), and fat mass, and optionally one or more genotypical data.

Genotypical data mean that it is also taken in consideration if certain mutations (e.g., single nucleotide polymorphisms SNP) are present, or if any other data related with genetic predisposition to a certain risk of disease or disorder, in particular related with metabolic disorders, are applicable to a subject.

In another particular embodiment of the first aspect, the isolated sample where the different markers are determined from a biofluid, in particular from one or more of blood, plasma, serum, and urine.

Although in the tables that follow particular markers are indicated to be determined in particular sample types, the skilled person in the art will understand that the most of markers can be measured in other sample types and will not consider compulsory to analyse a marker only a in a sample type to get a particular information from the same.

In a particular embodiment, the markers of clusters (a), (b), (c) are determined in an isolated sample of plasma or serum, and those markers of (d) and (e) are determined in an isolated sample of plasma, serum, or urine.

Thus, in a particular embodiment of the first aspect of the invention, the markers of cluster (a) determined in plasma are selected from glucose and propionylcarnitine, or both of them; the markers of cluster (b) determined in plasma are selected from one or more of LDL-cholesterol, Total cholesterol, HDL-cholesterol, Triglycerides, LPCs (total) and propionylcarnitine and combinations thereof, or all of them; the markers of cluster (c) determined in plasma are selected from MCP-1, TNFα, IL-6, IL-10, sICAM1, LPCs (total), LBP, sCD14, and combinations thereof, or all of them; the marker of cluster (d) determined in plasma is LBP; and the marker of cluster (e) determined in plasma is LDLox.

Thus, in a particular embodiment of the first aspect of the invention, the markers of cluster (a) determined in serum are selected from glutamate, uric acid, leptin, adiponectin, insulin, tyrosine, lactate, valine, leucine, isoleucine, phenylalanine, glutamine, combinations thereof, or all of them; the markers of cluster (b) determined in serum are selected from one or more of PUFAs (total), saturated fatty acids, MUFAs (total), linoleic acid, DHA, oleic acid, choline, 3-hydroxybutyrate, adiponectin, and combinations thereof, or all of them; the markers of cluster (c) determined in serum are selected from CRP, N-acetylglycoproteins, SFAs, DHA, linoleic acid, PUFAs, and combinations thereof, or all of them; the markers of cluster (d) determined in serum are selected from one or more of TMAO, choline, lactate, and combinations thereof, or all of them; and the markers of cluster (e) determined in serum are selected from one or more of uric acid, methionine, glycine, and combinations thereof, or all of them.

In also another particular embodiment, the markers of cluster (d) determined in urine are selected from one or more of TMA, betaine, DMA, dimethylglycine, succinate, acetate, and combinations thereof, or all of them; and the markers of cluster (e) determined in urine are selected from one or more of 8-iso-PGF2a, 8-OHdG urine, allantoin, betaine, pseudouridine, dimethylglycine, and combinations thereof, or all of them.

Following Tables A to E illustrate particular embodiments of the clusters of markers and in which isolated sample types are they measured and an assigned. Thus, in a particular embodiment of the method of the invention, the clusters (a) to (e) comprising the markers in the indicated isolated samples are determined in step (i).

TABLE A
Carbohydrate metabolism cluster

| | | | SIM | Normal/ |
|---|---|---|---|---|
| Biomarker | Reference | Biofluid | (P) | Reverse |
| Glucose | CAS: 50-99-7 | Plasma | 25 | Normal |
| HOMA-IR | | | 25 | Normal |
| Glutamate | CAS: 56-86-0 | Serum | 25 | Normal |
| Uric acid | CAS: 69-93-2 | Serum | 25 | Normal |
| Leptin | UniProt: A4D0Y8 | Serum | 15 | Normal |
| Adiponectin | UniProt: Q15848 | Serum | 15 | Reverse |
| Insulin | UniProt: P01308 | Serum | 15 | Normal |
| Tyrosine | CAS: 60-18-4 | Serum | 15 | Normal |
| Propionylcarnitine | CAS: 17298-37-2 | Plasma | 10 | Normal |
| Lactate | CAS: 50-21-5 | Serum | 10 | Normal |
| Valine | CAS: 72-18-4 | Serum | 10 | Normal |
| Leucine | CAS: 61-90-5 | Serum | 10 | Normal |
| Isoleucine | CAS: 73-32-5 | Serum | 10 | Normal |
| Phenylalanine | CAS: 63-91-2 | Serum | 10 | Normal |
| Glutamine | CAS: 56-85-9 | Serum | 8 | Reverse |

TABLE B
Lipid metabolism cluster

| | | | SIM | Normal/ |
|---|---|---|---|---|
| Biomarker | Reference | Biofluid | (P) | Reverse |
| LDL-cholesterol | | Plasma | 25 | Normal |
| Total cholesterol | | Plasma | 24 | Normal |
| PUFAs (total) | | Serum | 23 | Reverse |
| HDL-cholesterol | | Plasma | 21 | Reverse |
| Saturated fatty acids | | Serum | 20 | Normal |
| Triglycerides | | Plasma | 19 | Normal |
| MUFAs (total) | | Serum | 15 | Normal |
| LPCs(total) | | Plasma | 7 | Normal |
| Linoleic acid | CAS: 60-33-3 | Serum | 6 | Normal |
| DHA | CAS: 6217-54-5 | Serum | 4 | Reverse |
| Oleic acid | CAS: 112-80-1 | Serum | 4 | Normal |
| Choline | CAS: 62-49-7 | Serum | 4 | Normal |
| 3-hydroxybutyrate | CAS: 151-03-1 | Serum | 3 | Normal |
| Propionylcarnitine | CAS: 17298-37-2 | Plasma | 3 | Normal |
| Adiponectin | Uniprot: Q15848 | Serum | 2 | Reverse |
| Leptin | Uniprot: A4D0YB | Serum | 2 | Normal |

TABLE C
Inflammation cluster

| | | | SIM | Normal/ |
|---|---|---|---|---|
| Biomarker | Reference | Biofluid | (P) | Reverse |
| CRP | Uniprot: P02741 | Serum | 25 | Normal |
| N-acetylglycoproteins | | Serum | 25 | Normal |
| MCP-1 | Uniprot Q6UZ82 | Plasma | 25 | Normal |
| TNFα | Uniprot: P01375 | Plasma | 23 | Normal |
| IL-6 | Uniprot: P05231 | Plasma | 20 | Normal |
| IL-10 | Uniprot: P22301 | Plasma | 18 | Normal |
| SFAs | | Serum | 17 | Normal |
| sICAM1 | Uniprot: Q99930 | Plasma | 15 | Normal |
| LPCs (total) | | Plasma | 13 | Reverse |
| LBP | Uniprot: P18428 | Plasma | 13 | Normal |
| DHA | CAS: 6217-54-5 | Serum | 10 | Reverse |
| sCD14 | Soluble form of | Plasma | 10 | Normal |
| | Uniprot: P08571 | | | |
| Linoleic acid | CAS: 60-33-3 | Serum | 7 | Normal |
| PUFAs | | Serum | 5 | Reverse |

TABLE D
Microbiota cluster

| | | | SIM | Normal/ |
|---|---|---|---|---|
| Biomarker | Reference | Biofluid | (P) | Reverse |
| TMA | CAS: 75-50-3 | Urine | 25 | Normal |
| TMAO | CAS: 1184-78-7 | Serum | 25 | Normal |
| Betaine | CAS: 107-43-7 | Urine | 20 | Normal |
| Choline | CAS: 62-49-7 | Serum | 20 | Normal |
| DMA | CAS: 124-40-3 | Urine | 15 | Normal |
| Dimethylglycine | CAS: 1118-68-9 | Urine | 12 | Normal |
| LBP | Uniprot: P18428 | Plasma | 10 | Normal |
| Succinate | CAS: 110-15-6 | Urine | 8 | Reverse |
| Lactate | CAS: 113-21-3 | Serum | 8 | Normal |
| Acetate | CAS: 71-50-1 | Urine | 6 | Normal |

TABLE E
Oxidative stress cluster

| | | | SIM | Normal/ |
|---|---|---|---|---|
| Biomarker | Reference | Biofluid | (P) | Reverse |
| 8-iso-PGF2α | CAS: 27415-26-5 | Urine | 25 | Normal |
| 8-OHdG Urine | CAS: 3868-31-3 | Urine | 23 | Normal |
| LDLox | | Plasma | 21 | Normal |
| Uric acid | CAS: 69-93-2 | Serum | 21 | Reverse |
| Allantoin | CAS: 97-59-6 | Urine | 18 | Normal |
| Betaine | CAS: 107-43-7 | Urine | 15 | Normal |
| Pseudouridine | CAS: 1445-07-4 | Urine | 13 | Normal |
| Dimethylglycine | CAS: 1118-68-9 | Urine | 10 | Normal |
| Methionine | CAS: 63-68-31 | Serum | 10 | Reverse |
| Glycine | CAS: 56-40-61 | Serum | 5 | Reverse |

The skilled person in the art will know which kits assays and tests are the more appropriate for the analysis of each of the markers in the cited isolated samples.

The method of the invention is, in a particular embodiment, a computer-implemented method comprising at least one of the following steps (ii), (iii) and (iv). In a more particular embodiment comprising at least steps (ii) and (iii). More in particular, comprising at least steps (ii), (iii) and (iv).

In a particular embodiment the step (ii) gives an score to a cluster, which score results from a function considering the levels and/or concentrations of two or more markers determined in a sample as defined in step (i);

In a particular embodiment, a computer-implemented method in which at least one of the steps (ii) to (iv) are carried out in a computer, which computer comprises a memory that stores program instructions and a processor coupled to the memory to cause at least one of the steps (ii) to (iv).

In a more particular embodiment, the computer-implemented method includes carrying out all the steps (ii) to (iv) of the method of the invention.

In another particular embodiment of the method of the first aspect, in step (iv) a diet is recommended, which is selected from one or more diets appropriate for the control of one or more of the carbohydrate metabolism, the lipid metabolism, inflammation indicators status of the subject, the microbiota metabolism, and oxidative stress indicators of the subject.

In other words, independently of the diet chosen for each of the groups, which the skilled person in the art (i.e., nutritionist) will know, the recommended diet is a diet that includes more than one diet type for the control of one or more of the indicated metabolic clusters.

In even a more particular embodiment, the method further comprises the step of administering the selected diet. Thus, it is also herewith disclosed a method for the selection and administration of a diet for a subject based on health data, comprising:
(i) determining in an isolated sample of the subject two or more markers of two or more of the following metabolic clusters:
(a) carbohydrate metabolism;
(b) lipid metabolism;
(c) inflammation;
(d) microbiota metabolism; and
(e) oxidative stress;
(ii) giving a score to each cluster, which score results from a function considering the levels and/or concentrations of the two or more markers in the sample;
(iii) comparing the scores obtained for the two or more clusters (a) to (e) to determine a gradation between them;
(iv) recommending a diet for the subject considering the said gradation and optionally the individual score value in relation to a reference value for that score; and
(v) administering (i.e., thus treating) the subject with the selected diet.

This method for the selection and administration of the diet has, as particular embodiments, all those disclosed for the method of selection of the diet of the first aspect and indicated above.

Other particular embodiments of the method for the selection and administration of the diet relate to the type of diet finally administered, which is selected from one or more diets appropriate for the control of one or more of the carbohydrate metabolism, the lipid metabolism, inflammation indicators status of the subject, the microbiota metabolism, and oxidative stress indicators of the subject.

A first additional aspect of the invention corresponds to a computer-implemented method comprising at least one of the following steps:
(i) giving a score to a cluster, which score results from a function considering the levels and/or concentrations of two or more markers determined in a sample as defined in the first aspect;
(ii) comparing the scores obtained for the two or more clusters (a) to (e) to determine a gradation between them; and
(iii) recommending a diet for the subject considering the said gradation and optionally the individual score value in relation to a reference value for that score.

In a particular embodiment, the computer-implemented method comprises at least steps (i) and (ii). In a more particular embodiment, the computer implemented method comprises at least steps (i), (ii) and (iii).

As indicated, a second aspect of the invention corresponds to a data processing system for the selection of a diet for a subject based on health data, comprising:
a database that stores data determined in an isolated sample of the subject of the two or more markers of two or more of the following metabolic clusters:
(a) carbohydrate metabolism;
(b) lipid metabolism;
(c) inflammation;
(d) microbiota metabolism; and
(e) oxidative stress;
a memory that stores program instructions, including program instructions that are capable of implementing: (i) a function to get a score of the metabolic clusters from the levels and/or concentrations of the two or more markers in the sample; (ii) a comparison of the scores to determine a gradation between them; and (iii) a diet selection for the subject based on the score of the metabolic clusters and their comparison; and
a processor, coupled to the database and the memory, that when executing the program instructions causes the function to get a score of the metabolic clusters from the levels and/or concentrations of the two or more markers in the sample, causes the comparison of the scores to determine a gradation between them, and causes a diet selection for the subject based on the score of the metabolic clusters and their comparison.

In a particular embodiment, the system is adapted to perform the method according to the first aspect and any of its disclosed particular embodiments.

Thus, in a more particular embodiment of the system, the system implements step (ii) by comparing the scores and determining the higher of them; and implements step (iii) of the method of the first aspect by selecting a diet based on this higher of the scores.

In other words, the second aspect corresponds to a data processing system comprising means for carrying out the steps of the computer-implemented method as defined in the first additional aspect.

In a particular embodiment, means comprise the database, the memory and the processor as defined above.

In another particular embodiment, the system is adapted to perform the computer-implemented method according to the first additional aspect and any of its disclosed particular embodiments.

The practical applications of the method, the computer-implemented method and the system of the invention includes, among others, the nutritional recommendation based on food groups and the integration of these recommendations within a food product catalogue. Another practical implementation of the method, computer-implemented method and system is the integration of the recommendations within a particular software for nutritionists and/or for the subjects (e.g., web app). Also, another practical implementation of the method, computer-implemented method and system of the invention is a dashboard (i.e., visual display of the data) or a web page to get information from the user (i.e., subject) to finally inform him or her about the personal nutritional recommendation.

The system of the second aspect of invention is improved with the implementation of artificial intelligence, by means of neuronal networks that include optimized information to improve in turn the performance of the method and system of the invention.

The in vitro methods of the invention provide relevant information for nutritional recommendations or for the selection of a diet for a subject. In one embodiment, the methods of the invention further comprise the steps of (v) collecting the information about the recommendation, and (vi) saving the information in a data carrier.

In the sense of the invention a “data carrier” is to be understood as any means that contain meaningful information data for the selection of the diet, such as paper. For example, the carrier may comprise a storage medium, such as a ROM, for example a CD ROM or a semiconductor ROM, or a magnetic recording medium, for example a floppy disc or hard disk. Further, the carrier may be a transmissible carrier such as an electrical or optical signal, which may be conveyed via electrical or optical cable or by radio or other means. When the diagnosis/prognosis data are embodied in a signal that may be conveyed directly by a cable or other device or means, the carrier may be constituted by such cable or other device or means. Other carriers relate to USB devices and computer archives. Examples of suitable data carrier are paper, CDs, USB, computer archives in PCs, or sound registration with the same information.

A second additional aspect of the invention corresponds to a computer program comprising instructions which, when the program is executed by a computer, cause the computer to carry out the steps of the method as defined in the first and/or the first additional aspect of the invention.

A third additional aspect of the invention corresponds to a computer-readable data carrier having stored thereon the computer program of the second additional aspect.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

TABLE F
Carbohydrate metabolism cluster: ranges for the proportions between biomarkers' SIM values.

| | SIM(cluster)ₙ/SIM(cluster)_{Glucose} | | SIM(cluster)ₙ/SIM(cluster)_{HOMA-IR} | |
|---|---|---|---|---|
| n | Lower limit | Upper limit | Lower limit | Upper limit |
| Glucose | 1 | | (25 − 25*X)/(25 + 25*X) | (25 + 25*X)/(25 − 25*X) |
| HOMA-IR | (25 − 25*X)/(25 + 25*X) | (25 + 25*X)/(25 − 25*X) | 1 | |
| Glutamate | (25 − 25*X)/(25 + 25*X) | (25 + 25*X)/(25 − 25*X) | (25 − 25*X)/(25 + 25*X) | (25 + 25*X)/(25 − 25*X) |
| Uric acid | (25 − 25*X)/(25 + 25*X) | (25 + 25*X)/(25 − 25*X) | (25 − 25*X)/(25 + 25*X) | (25 + 25*X)/(25 − 25*X) |
| Leptin | (15 − 15*X)/(25 + 25*X) | (15 + 15*X)/(25 − 25*X) | (15 − 15*X)/(25 + 25*X) | (15 + 15*X)/(25 − 25*X) |
| Adiponectin | (15 − 15*X)/(25 + 25*X) | (15 + 15*X)/(25 − 25*X) | (15 − 15*X)/(25 + 25*X) | (15 + 15*X)/(25 − 25*X) |
| Insulin | (15 − 15*X)/(25 + 25*X) | (15 + 15*X)/(25 − 25*X) | (15 − 15*X)/(25 + 25*X) | (15 + 15*X)/(25 − 25*X) |
| Tyrosine | (15 − 15*X)/(25 + 25*X) | (15 + 15*X)/(25 − 25*X) | (15 − 15*X)/(25 + 25*X) | (15 + 15*X)/(25 − 25*X) |
| Propionylcarnitine | (10 − 10*X)/(25 + 25*X) | (10 + 10*X)/(25 − 25*X) | (10 − 10*X)/(25 + 25*X) | (10 + 10*X)/(25 − 25*X) |
| Lactate | (10 − 10*X)/(25 + 25*X) | (10 + 10*X)/(25 − 25*X) | (10 − 10*X)/(25 + 25*X) | (10 + 10*X)/(25 − 25*X) |
| Valine | (10 − 10*X)/(25 + 25*X) | (10 + 10*X)/(25 − 25*X) | (10 − 10*X)/(25 + 25*X) | (10 + 10*X)/(25 − 25*X) |
| Leucine | (10 − 10*X)/(25 + 25*X) | (10 + 10*X)/(25 − 25*X) | (10 − 10*X)/(25 + 25*X) | (10 + 10*X)/(25 − 25*X) |
| Isoleucine | (10 − 10*X)/(25 + 25*X) | (10 + 10*X)/(25 − 25*X) | (10 − 10*X)/(25 + 25*X) | (10 + 10*X)/(25 − 25*X) |
| Phenylalanine | (10 − 10*X)/(25 + 25*X) | (10 + 10*X)/(25 − 25*X) | (10 − 10*X)/(25 + 25*X) | (10 + 10*X)/(25 − 25*X) |
| Glutamine | (8 − 8*X)/(25 + 25*X) | (8 + 8*X)/(25 − 25*X) | (8 − 8*X)/(25 + 25*X) | (8 + 8*X)/(25 − 25*X) |
| | SIM(cluster)ₙ/SIM(cluster)_{Glutamate} | | SIM(cluster)ₙ/SIM(cluster)_{Uric acid} | |
| n | Lower limit | Upper limit | Lower limit | Upper limit |
| Glucose | (25 − 25*X)/(25 + 25*X) | (25 + 25*X)/(25 − 25*X) | (25 − 25*X)/(25 + 25*X) | (25 + 25*X)/(25 − 25*X) |
| HOMA-IR | (25 − 25*X)/(25 + 25*X) | (25 + 25*X)/(25 − 25*X) | (25 − 25*X)/(25 + 25*X) | (25 + 25*X)/(25 − 25*X) |
| Glutamate | 1 | | (25 − 25*X)/(25 + 25*X) | (25 + 25*X)/(25 − 25*X) |
| Uric acid | (25 − 25*X)/(25 + 25*X) | (25 + 25*X)/(25 − 25*X) | 1 | |
| Leptin | (15 − 15*X)/(25 + 25*X) | (15 + 15*X)/(25 − 25*X) | (15 − 15*X)/(25 + 25*X) | (15 + 15*X)/(25 − 25*X) |
| Adiponectin | (15 − 15*X)/(25 + 25*X) | (15 + 15*X)/(25 − 25*X) | (15 − 15*X)/(25 + 25*X) | (15 + 15*X)/(25 − 25*X) |
| Insulin | (15 − 15*X)/(25 + 25*X) | (15 + 15*X)/(25 − 25*X) | (15 − 15*X)/(25 + 25*X) | (15 + 15*X)/(25 − 25*X) |
| Tyrosine | (15 − 15*X)/(25 + 25*X) | (15 + 15*X)/(25 − 25*X) | (15 − 15*X)/(25 + 25*X) | (15 + 15*X)/(25 − 25*X) |
| Propionylcarnitine | (10 − 10*X)/(25 + 25*X) | (10 + 10*X)/(25 − 25*X) | (10 − 10*X)/(25 + 25*X) | (10 + 10*X)/(25 − 25*X) |
| Lactate | (10 − 10*X)/(25 + 25*X) | (10 + 10*X)/(25 − 25*X) | (10 − 10*X)/(25 + 25*X) | (10 + 10*X)/(25 − 25*X) |
| Valine | (10 − 10*X)/(25 + 25*X) | (10 + 10*X)/(25 − 25*X) | (10 − 10*X)/(25 + 25*X) | (10 + 10*X)/(25 − 25*X) |
| Leucine | (10 − 10*X)/(25 + 25*X) | (10 + 10*X)/(25 − 25*X) | (10 − 10*X)/(25 + 25*X) | (10 + 10*X)/(25 − 25*X) |
| Isoleucine | (10 − 10*X)/(25 + 25*X) | (10 + 10*X)/(25 − 25*X) | (10 − 10*X)/(25 + 25*X) | (10 + 10*X)/(25 − 25*X) |
| Phenylalanine | (10 − 10*X)/(25 + 25*X) | (10 + 10*X)/(25 − 25*X) | (10 − 10*X)/(25 + 25*X) | (10 + 10*X)/(25 − 25*X) |
| Glutamine | (8 − 8*X)/(25 + 25*X) | (8 + 8*X)/(25 − 25*X) | (8 − 8*X)/(25 + 25*X) | (8 + 8*X)/(25 − 25*X) |
| | SIM(cluster)ₙ/SIM(cluster)_{Leptin} | | SIM(cluster)ₙ/SIM(cluster)_{Adiponectin} | |
| n | Lower limit | Upper limit | Lower limit | Upper limit |
| Glucose | (25 − 25*X)/(15 + 15*X) | (25 + 25*X)/(15 − 15*X) | (25 − 25*X)/(15 + 15*X) | (25 + 25*X)/(15 − 15*X) |
| HOMA-IR | (25 − 25*X)/(15 + 15*X) | (25 + 25*X)/(15 − 15*X) | (25 − 25*X)/(15 + 15*X) | (25 + 25*X)/(15 − 15*X) |
| Glutamate | (25 − 25*X)/(15 + 15*X) | (25 + 25*X)/(15 − 15*X) | (25 − 25*X)/(15 + 15*X) | (25 + 25*X)/(15 − 15*X) |
| Uric acid | (25 − 25*X)/(15 + 15*X) | (25 + 25*X)/(15 − 15*X) | (25 − 25*X)/(15 + 15*X) | (25 + 25*X)/(15 − 15*X) |
| Leptin | 1 | | (15 − 15*X)/(15 + 15*X) | (15 + 15*X)/(15 − 15*X) |
| Adiponectin | (15 − 15*X)/(15 + 15*X) | (15 + 15*X)/(15 − 15*X) | 1 | |
| Insulin | (15 − 15*X)/(15 + 15*X) | (15 + 15*X)/(15 − 15*X) | (15 − 15*X)/(15 + 15*X) | (15 + 15*X)/(15 − 15*X) |
| Tyrosine | (15 − 15*X)/(15 + 15*X) | (15 + 15*X)/(15 − 15*X) | (15 − 15*X)/(15 + 15*X) | (15 + 15*X)/(15 − 15*X) |
| Propionylcarnitine | (10 − 10*X)/(15 + 15*X) | (10 + 10*X)/(15 − 15*X) | (10 − 10*X)/(15 + 15*X) | (10 + 10*X)/(15 − 15*X) |
| Lactate | (10 − 10*X)/(15 + 15*X) | (10 + 10*X)/(15 − 15*X) | (10 − 10*X)/(15 + 15*X) | (10 + 10*X)/(15 − 15*X) |
| Valine | (10 − 10*X)/(15 + 15*X) | (10 + 10*X)/(15 − 15*X) | (10 − 10*X)/(15 + 15*X) | (10 + 10*X)/(15 − 15*X) |
| Leucine | (10 − 10*X)/(15 + 15*X) | (10 + 10*X)/(15 − 15*X) | (10 − 10*X)/(15 + 15*X) | (10 + 10*X)/(15 − 15*X) |
| Isoleucine | (10 − 10*X)/(15 + 15*X) | (10 + 10*X)/(15 − 15*X) | (10 − 10*X)/(15 + 15*X) | (10 + 10*X)/(15 − 15*X) |
| Phenylalanine | (10 − 10*X)/(15 + 15*X) | (10 + 10*X)/(15 − 15*X) | (10 − 10*X)/(15 + 15*X) | (10 + 10*X)/(15 − 15*X) |
| Glutamine | (8 − 8*X)/(15 + 15*X) | (8 + 8*X)/(15 − 15*X) | (8 − 8*X)/(15 + 15*X) | (8 + 8*X)/(15 − 15*X) |
| | SIM(cluster)ₙ/SIM(cluster)_{Insulin} | | SIM(cluster)ₙ/SIM(cluster)_{Tyrosine} | |
| n | Lower limit | Upper limit | Lower limit | Upper limit |
| Glucose | (25 − 25*X)/(15 + 15*X) | (25 + 25*X)/(15 − 15*X) | (25 − 25*X)/(15 + 15*X) | (25 + 25*X)/(15 − 15*X) |
| HOMA-IR | (25 − 25*X)/(15 + 15*X) | (25 + 25*X)/(15 − 15*X) | (25 − 25*X)/(15 + 15*X) | (25 + 25*X)/(15 − 15*X) |
| Glutamate | (25 − 25*X)/(15 + 15*X) | (25 + 25*X)/(15 − 15*X) | (25 − 25*X)/(15 + 15*X) | (25 + 25*X)/(15 − 15*X) |
| Uric acid | (25 − 25*X)/(15 + 15*X) | (25 + 25*X)/(15 − 15*X) | (25 − 25*X)/(15 + 15*X) | (25 + 25*X)/(15 − 15*X) |
| Leptin | (15 − 15*X)/(15 + 15*X) | (15 + 15*X)/(15 − 15*X) | (15 − 15*X)/(15 + 15*X) | (15 + 15*X)/(15 − 15*X) |
| Adiponectin | (15 − 15*X)/(15 + 15*X) | (15 + 15*X)/(15 − 15*X) | (15 − 15*X)/(15 + 15*X) | (15 + 15*X)/(15 − 15*X) |
| Insulin | 1 | | (15 − 15*X)/(15 + 15*X) | (15 + 15*X)/(15 − 15*X) |
| Tyrosine | (15 − 15*X)/(15 + 15*X) | (15 + 15*X)/(15 − 15*X) | 1 | |
| Propionylcarnitine | (10 − 10*X)/(15 + 15*X) | (10 + 10*X)/(15 − 15*X) | (10 − 10*X)/(15 + 15*X) | (10 + 10*X)/(15 − 15*X) |
| Lactate | (10 − 10*X)/(15 + 15*X) | (10 + 10*X)/(15 − 15*X) | (10 − 10*X)/(15 + 15*X) | (10 + 10*X)/(15 − 15*X) |
| Valine | (10 − 10*X)/(15 + 15*X) | (10 + 10*X)/(15 − 15*X) | (10 − 10*X)/(15 + 15*X) | (10 + 10*X)/(15 − 15*X) |
| Leucine | (10 − 10*X)/(15 + 15*X) | (10 + 10*X)/(15 − 15*X) | (10 − 10*X)/(15 + 15*X) | (10 + 10*X)/(15 − 15*X) |
| Isoleucine | (10 − 10*X)/(15 + 15*X) | (10 + 10*X)/(15 − 15*X) | (10 − 10*X)/(15 + 15*X) | (10 + 10*X)/(15 − 15*X) |
| Phenylalanine | (10 − 10*X)/(15 + 15*X) | (10 + 10*X)/(15 − 15*X) | (10 − 10*X)/(15 + 15*X) | (10 + 10*X)/(15 − 15*X) |
| Glutamine | (8 − 8*X)/(15 + 15*X) | (8 + 8*X)/(15 − 15*X) | (8 − 8*X)/(15 + 15*X) | (8 + 8*X)/(15 − 15*X) |
| | SIM(cluster)ₙ/SIM(cluster)_{Propionylcarnitine} | | SIM(cluster)ₙ/SIM(cluster)_{Lactate} | |
| n | Lower limit | Upper limit | Lower limit | Upper limit |
| Glutamate | (25 − 25*X)/(10 + 10*X) | (25 + 25*X)/(10 − 10*X) | (25 − 25*X)/(10 + 10*X) | (25 + 25*X)/(10 − 10*X) |
| HOMA-IR | (25 − 25*X)/(10 + 10*X) | (25 + 25*X)/(10 − 10*X) | (25 − 25*X)/(10 + 10*X) | (25 + 25*X)/(10 − 10*X) |
| Glutamate | (25 − 25*X)/(10 + 10*X) | (25 + 25*X)/(10 − 10*X) | (25 − 25*X)/(10 + 10*X) | (25 + 25*X)/(10 − 10*X) |
| Uric acid | (25 − 25*X)/(10 + 10*X) | (25 + 25*X)/(10 − 10*X) | (25 − 25*X)/(10 + 10*X) | (25 + 25*X)/(10 − 10*X) |
| Leptin | (15 − 15*X)/(10 + 10*X) | (15 + 15*X)/(10 − 10*X) | (15 − 15*X)/(10 + 10*X) | (15 + 15*X)/(10 − 10*X) |
| Adiponectin | (15 − 15*X)/(10 + 10*X) | (15 + 15*X)/(10 − 10*X) | (15 − 15*X)/(10 + 10*X) | (15 + 15*X)/(10 − 10*X) |
| Insulin | (15 − 15*X)/(10 + 10*X) | (15 + 15*X)/(10 − 10*X) | (15 − 15*X)/(10 + 10*X) | (15 + 15*X)/(10 − 10*X) |
| Tyrosine | (15 − 15*X)/(10 + 10*X) | (15 + 15*X)/(10 − 10*X) | (15 − 15*X)/(10 + 10*X) | (15 + 15*X)/(10 − 10*X) |
| Propionylcarnitine | 1 | | (10 − 10*X)/(10 + 10*X) | (10 + 10*X)/(10 − 10*X) |
| Lactate | (10 − 10*X)/(10 + 10*X) | (10 + 10*X)/(10 − 10*X) | 1 | |
| Valine | (10 − 10*X)/(10 + 10*X) | (10 + 10*X)/(10 − 10*X) | (10 − 10*X)/(10 + 10*X) | (10 + 10*X)/(10 − 10*X) |
| Leucine | (10 − 10*X)/(10 + 10*X) | (10 + 10*X)/(10 − 10*X) | (10 − 10*X)/(10 + 10*X) | (10 + 10*X)/(10 − 10*X) |
| Isoleucine | (10 − 10*X)/(10 + 10*X) | (10 + 10*X)/(10 − 10*X) | (10 − 10*X)/(10 + 10*X) | (10 + 10*X)/(10 − 10*X) |
| Phenylalanine | (10 − 10*X)/(10 + 10*X) | (10 + 10*X)/(10 − 10*X) | (10 − 10*X)/(10 + 10*X) | (10 + 10*X)/(10 − 10*X) |
| Glutamine | (8 − 8*X)/(10 + 10*X) | (8 + 8*X)/(10 − 10*X) | (8 − 8*X)/(10 + 10*X) | (8 + 8*X)/(10 − 10*X) |
| | SIM(cluster)ₙ/SIM(cluster)_{Valine} | | SIM(cluster)ₙ/SIM(cluster)_{Leucine} | |
| n | Lower limit | Upper limit | Lower limit | Upper limit |
| Glucose | (25 − 25*X)/(10 + 10*X) | (25 + 25*X)/(10 − 10*X) | (25 − 25*X)/(10 + 10*X) | (25 + 25*X)/(10 − 10*X) |
| HOMA-IR | (25 − 25*X)/(10 + 10*X) | (25 + 25*X)/(10 − 10*X) | (25 − 25*X)/(10 + 10*X) | (25 + 25*X)/(10 − 10*X) |
| Glutamate | (25 − 25*X)/(10 + 10*X) | (25 + 25*X)/(10 − 10*X) | (25 − 25*X)/(10 + 10*X) | (25 + 25*X)/(10 − 10*X) |
| Uric acid | (25 − 25*X)/(10 + 10*X) | (25 + 25*X)/(10 − 10*X) | (25 − 25*X)/(10 + 10*X) | (25 + 25*X)/(10 − 10*X) |
| Leptin | (15 − 15*X)/(10 + 10*X) | (15 + 15*X)/(10 − 10*X) | (15 − 15*X)/(10 + 10*X) | (15 + 15*X)/(10 − 10*X) |
| Adiponectin | (15 − 15*X)/(10 + 10*X) | (15 + 15*X)/(10 − 10*X) | (15 − 15*X)/(10 + 10*X) | (15 + 15*X)/(10 − 10*X) |
| Insulin | (15 − 15*X)/(10 + 10*X) | (15 + 15*X)/(10 − 10*X) | (15 − 15*X)/(10 + 10*X) | (15 + 15*X)/(10 − 10*X) |
| Tyrosine | (15 − 15*X)/(10 + 10*X) | (15 + 15*X)/(10 − 10*X) | (15 − 15*X)/(10 + 10*X) | (15 + 15*X)/(10 − 10*X) |
| Propionylcarnitine | (10 − 10*X)/(10 + 10*X) | (10 + 10*X)/(10 − 10*X) | (10 − 10*X)/(10 + 10*X) | (10 + 10*X)/(10 − 10*X) |
| Lactate | (10 − 10*X)/(10 + 10*X) | (10 + 10*X)/(10 − 10*X) | (10 − 10*X)/(10 + 10*X) | (10 + 10*X)/(10 − 10*X) |
| Valine | 1 | | (10 − 10*X)/(10 + 10*X) | (10 + 10*X)/(10 − 10*X) |
| Leucine | (10 − 10*X)/(10 + 10*X) | (10 + 10*X)/(10 − 10*X) | 1 | |
| Isoleucine | (10 − 10*X)/(10 + 10*X) | (10 + 10*X)/(10 − 10*X) | (10 − 10*X)/(10 + 10*X) | (10 + 10*X)/(10 − 10*X) |
| Phenylalanine | (10 − 10*X)/(10 + 10*X) | (10 + 10*X)/(10 − 10*X) | (10 − 10*X)/(10 + 10*X) | (10 + 10*X)/(10 − 10*X) |
| Glutamine | (8 − 8*X)/(10 + 10*X) | (8 + 8*X)/(10 − 10*X) | (8 − 8*X)/(10 + 10*X) | (8 + 8*X)/(10 − 10*X) |
| | SIM(cluster)ₙ/SIM(cluster)_{Isoleucine} | | SIM(cluster)ₙ/SIM(cluster)_{Phenylalanine} | |
| n | Lower limit | Upper limit | Lower limit | Upper limit |
| Glucose | (25 − 25*X)/(10 + 10*X) | (25 + 25*X)/(10 − 10*X) | (25 − 25*X)/(10 + 10*X) | (25 + 25*X)/(10 − 10*X) |
| HOMA-IR | (25 − 25*X)/(10 + 10*X) | (25 + 25*X)/(10 − 10*X) | (25 − 25*X)/(10 + 10*X) | (25 + 25*X)/(10 − 10*X) |
| Glutamate | (25 − 25*X)/(10 + 10*X) | (25 + 25*X)/(10 − 10*X) | (25 − 25*X)/(10 + 10*X) | (25 + 25*X)/(10 − 10*X) |
| Uric acid | (25 − 25*X)/(10 + 10*X) | (25 + 25*X)/(10 − 10*X) | (25 − 25*X)/(10 + 10*X) | (25 + 25*X)/(10 − 10*X) |
| Leptin | (15 − 15*X)/(10 + 10*X) | (15 + 15*X)/(10 − 10*X) | (15 − 15*X)/(10 + 10*X) | (15 + 15*X)/(10 − 10*X) |
| Adiponectin | (15 − 15*X)/(10 + 10*X) | (15 + 15*X)/(10 − 10*X) | (15 − 15*X)/(10 + 10*X) | (15 + 15*X)/(10 − 10*X) |
| Insulin | (15 − 15*X)/(10 + 10*X) | (15 + 15*X)/(10 − 10*X) | (15 − 15*X)/(10 + 10*X) | (15 + 15*X)/(10 − 10*X) |
| Tyrosine | (15 − 15*X)/(10 + 10*X) | (15 + 15*X)/(10 − 10*X) | (15 − 15*X)/(10 + 10*X) | (15 + 15*X)/(10 − 10*X) |
| Propionylcarnitine | (10 − 10*X)/(10 + 10*X) | (10 + 10*X)/(10 − 10*X) | (10 − 10*X)/(10 + 10*X) | (10 + 10*X)/(10 − 10*X) |
| Lactate | (10 − 10*X)/(10 + 10*X) | (10 + 10*X)/(10 − 10*X) | (10 − 10*X)/(10 + 10*X) | (10 + 10*X)/(10 − 10*X) |
| Valine | (10 − 10*X)/(10 + 10*X) | (10 + 10*X)/(10 − 10*X) | (10 − 10*X)/(10 + 10*X) | (10 + 10*X)/(10 − 10*X) |
| Leucine | (10 − 10*X)/(10 + 10*X) | (10 + 10*X)/(10 − 10*X) | (10 − 10*X)/(10 + 10*X) | (10 + 10*X)/(10 − 10*X) |
| Isoleucine | 1 | | (10 − 10*X)/(10 + 10*X) | (10 + 10*X)/(10 − 10*X) |
| Phenylalanine | (10 − 10*X)/(10 + 10*X) | (10 + 10*X)/(10 − 10*X) | 1 | |
| Glutamine | (8 − 8*X)/(10 + 10*X) | (8 + 8*X)/(10 − 10*X) | (8 − 8*X)/(10 + 10*X) | (8 + 8*X)/(10 − 10*X) |

SIM(cluster)ₙ/SIM(cluster)_{Glutamine}

| n | Lower limit | Upper limit |
|---|---|---|
| Glucose | (25 − 25*X)/(8 + 8*X) | (25 + 25*X)/(8 − 8*X) |
| HOMA-IR | (25 − 25*X)/(8 + 8*X) | (25 + 25*X)/(8 − 8*X) |
| Glutamate | (25 − 25*X)/(8 + 8*X) | (25 + 25*X)/(8 − 8*X) |
| Uric acid | (25 − 25*X)/(8 + 8*X) | (25 + 25*X)/(8 − 8*X) |
| Leptin | (15 − 15*X)/(8 + 8*X) | (15 + 15*X)/(8 − 8*X) |
| Adiponectin | (15 − 15*X)/(8 + 8*X) | (15 + 15*X)/(8 − 8*X) |
| Insulin | (15 − 15*X)/(8 + 8*X) | (15 + 15*X)/(8 − 8*X) |
| Tyrosine | (15 − 15*X)/(8 + 8*X) | (15 + 15*X)/(8 − 8*X) |
| Propionylcarnitine | (10 − 10*X)/(8 + 8*X) | (10 + 10*X)/(8 − 8*X) |
| Lactate | (10 − 10*X)/(8 + 8*X) | (10 + 10*X)/(8 − 8*X) |
| Valine | (10 − 10*X)/(8 + 8*X) | (10 + 10*X)/(8 − 8*X) |
| Leucine | (10 − 10*X)/(8 + 8*X) | (10 + 10*X)/(8 − 8*X) |
| Isoleucine | (10 − 10*X)/(8 + 8*X) | (10 + 10*X)/(8 − 8*X) |
| Phenylalanine | (10 − 10*X)/(8 + 8*X) | (10 + 10*X)/(8 − 8*X) |

| | |
|---|---|
| Glutamine | 1 |

Wherein “SIM(cluster)” is the Score of individual marker (punctuation) of a particular marker determined in the isolated sample for a particular cluster; “n” is one specific biomarker of the cluster; and “cluster” is carbohydrate metabolism cluster.

TABLE G
Lipid metabolism cluster: ranges for the proportions between biomarkers' SIM values.

| | SIM(cluster)ₙ/SIM(cluster)_{LDL-cholesterol} | | SIM(cluster)ₙ/SIM(cluster)_{Total cholesterol} | |
|---|---|---|---|---|
| n | Lower limit | Upper limit | Lower limit | Upper limit |
| LDL-cholesterol | 1 | | (25 − 25*X)/(24 + 24*X) | (25 + 25*X)/(24 − 24*X) |
| Total cholesterol | (24 − 24*X)/(25 + 25*X) | (24 + 24*X)/(25 − 25*X) | 1 | |
| PUFAs (total) | (23 − 23*X)/(25 + 25*X) | (23 + 23*X)/(25 − 25*X) | (23 − 23*X)/(24 + 24*X) | (23 + 23*X)/(24 − 24*X) |
| HDL-cholesterol | (21 − 21*X)/(25 + 25*X) | (21 + 21*X)/(25 − 25*X) | (21 − 21*X)/(24 + 24*X) | (21 + 21*X)/(24 − 24*X) |
| Saturated fatty | (20 − 20*X)/(25 + 25*X) | (20 + 20*X)/(25 − 25*X) | (20 − 20*X)/(24 + 24*X) | (20 + 20*X)/(24 − 24*X) |
| acids | | | | |
| Triglycerides | (19 − 19*X)/(25 + 25*X) | (19 + 19*X)/(25 − 25*X) | (19 − 19*X)/(24 + 24*X) | (19 + 19*X)/(24 − 24*X) |
| MUFAs (total) | (15 − 15*X)/(25 + 25*X) | (15 + 15*X)/(25 − 25*X) | (15 − 15*X)/(24 + 24*X) | (15 + 15*X)/(24 − 24*X) |
| LPCs (total) | (7 − 7*X)/(25 + 25*X) | (7 + 7*X)/(25 − 25*X) | (7 − 7*X)/(24 + 24*X) | (7 + 7*X)/(24 − 24*X) |
| Linoleic acid | (6 − 6*X)/(25 + 25*X) | (6 + 6*X)/(25 − 25*X) | (6 − 6*X)/(24 + 24*X) | (6 + 6*X)/(24 − 24*X) |
| DHA | (4 − 4*X)/(25 + 25*X) | (4 + 4*X)/(25 − 25*X) | (4 − 4*X)/(24 + 24*X) | (4 + 4*X)/(24 − 24*X) |
| Oleic acid | (4 − 4*X)/(25 + 25*X) | (4 + 4*X)/(25 − 25*X) | (4 − 4*X)/(24 + 24*X) | (4 + 4*X)/(24 − 24*X) |
| Choline | (4 − 4*X)/(25 + 25*X) | (4 + 4*X)/(25 − 25*X) | (4 − 4*X)/(24 + 24*X) | (4 + 4*X)/(24 − 24*X) |
| 3-hydroxybutyrate | (3 − 3*X)/(25 + 25*X) | (3 + 3*X)/(25 − 25*X) | (3 − 3*X)/(24 + 24*X) | (3 + 3*X)/(24 − 24*X) |
| Propionylcarnitine | (3 − 3*X)/(25 + 25*X) | (3 + 3*X)/(25 − 25*X) | (3 − 3*X)/(24 + 24*X) | (3 + 3*X)/(24 − 24*X) |
| Adiponectin | (2 − 2*X)/(25 + 25*X) | (2 + 2*X)/(25 − 25*X) | (2 − 2*X)/(24 + 24*X) | (2 + 2*X)/(24 − 24*X) |
| Leptin | (2 − 2*X)/(25 + 25*X) | (2 + 2*X)/(25 − 25*X) | (2 − 2*X)/(24 + 24*X) | (2 + 2*X)/(24 − 24*X) |
| | SIM(cluster)ₙ/SIM(cluster)_{PUFAs (total)} | | SIM(cluster)ₙ/SIM(cluster)_{HDL-cholesterol} | |
| n | Lower limit | Upper limit | Lower limit | Upper limit |
| LDL-cholesterol | (25 − 25*X)/(23 + 23*X) | (25 + 25*X)/(23 − 23*X) | (25 − 25*X)/(21 + 21*X) | (25 + 25*X)/(21 − 21*X) |
| Total cholesterol | (24 − 24*X)/(23 + 23*X) | (24 + 24*X)/(23 − 23*X) | (24 − 24*X)/(21 + 21*X) | (24 + 24*X)/(21 − 21*X) |
| PUFAs (total) | 1 | | (23 − 23*X)/(21 + 21*X) | (23 + 23*X)/(21 − 21*X) |
| HDL-cholesterol | (21 − 21*X)/(23 + 23*X) | (21 + 21*X)/(23 − 23*X) | 1 | |
| Saturated fatty | (20 − 20*X)/(23 + 23*X) | (20 + 20*X)/(23 − 23*X) | (20 − 20*X)/(21 + 21*X) | (20 + 20*X)/(21 − 21*X) |
| acids | | | | |
| Triglycerides | (19 − 19*X)/(23 + 23*X) | (19 + 19*X)/(23 − 23*X) | (19 − 19*X)/(21 + 21*X) | (19 + 19*X)/(21 − 21*X) |
| MUFAs (total) | (15 − 15*X)/(23 + 23*X) | (15 + 15*X)/(23 − 23*X) | (15 − 15*X)/(21 + 21*X) | (15 + 15*X)/(21 − 21*X) |
| LPCs (total) | (7 − 7*X)/(23 + 23*X) | (7 + 7*X)/(23 − 23*X) | (7 − 7*X)/(21 + 21*X) | (7 + 7*X)/(21 − 21*X) |
| Linoleic acid | (6 − 6*X)/(23 + 23*X) | (6 + 6*X)/(23 − 23*X) | (6 − 6*X)/(21 + 21*X) | (6 + 6*X)/(21 − 21*X) |
| DHA | (4 − 4*X)/(23 + 23*X) | (4 + 4*X)/(23 − 23*X) | (4 − 4*X)/(21 + 21*X) | (4 + 4*X)/(21 − 21*X) |
| Oleic acid | (4 − 4*X)/(23 + 23*X) | (4 + 4*X)/(23 − 23*X) | (4 − 4*X)/(21 + 21*X) | (4 + 4*X)/(21 − 21*X) |
| Choline | (4 − 4*X)/(23 + 23*X) | (4 + 4*X)/(23 − 23*X) | (4 − 4*X)/(21 + 21*X) | (4 + 4*X)/(21 − 21*X) |
| 3-hydroxybutyrate | (3 − 3*X)/(23 + 23*X) | (3 + 3*X)/(23 − 23*X) | (3 − 3*X)/(21 + 21*X) | (3 + 3*X)/(21 − 21*X) |
| Propionylcarnitine | (3 − 3*X)/(23 + 23*X) | (3 + 3*X)/(23 − 23*X) | (3 − 3*X)/(21 + 21*X) | (3 + 3*X)/(21 − 21*X) |
| Adiponectin | (2 − 2*X)/(23 + 23*X) | (2 + 2*X)/(23 − 23*X) | (2 − 2*X)/(21 + 21*X) | (2 + 2*X)/(21 − 21*X) |
| Leptin | (2 − 2*X)/(23 + 23*X) | (2 + 2*X)/(23 − 23*X) | (2 − 2*X)/(21 + 21*X) | (2 + 2*X)/(21 − 21*X) |
| | SIM(cluster)ₙ/SIM(cluster)_{Saturated fatty acids} | | SIM(cluster)ₙ/SIM(cluster)_{Triglycerides} | |
| n | Lower limit | Upper limit | Lower limit | Upper limit |
| LDL-cholesterol | (25 − 25*X)/(20 + 20*X) | (25 + 25*X)/(20 − 20*X) | (25 − 25*X)/(19 + 19*X) | (25 + 25*X)/(19 − 19*X) |
| Total cholesterol | (24 − 24*X)/(20 + 20*X) | (24 + 24*X)/(20 − 20*X) | (24 − 24*X)/(19 + 19*X) | (24 + 24*X)/(19 − 19*X) |
| PUFAs (total) | (23 − 23*X)/(20 + 20*X) | (23 + 23*X)/(20 − 20*X) | (23 − 23*X)/(19 + 19*X) | (23 + 23*X)/(19 − 19*X) |
| HDL-cholesterol | (21 − 21*X)/(20 + 20*X) | (21 + 21*X)/(20 − 20*X) | (21 − 21*X)/(19 + 19*X) | (21 + 21*X)/(19 − 19*X) |
| Saturated fatty | 1 | | (20 − 20*X)/(19 + 19*X) | (20 + 20*X)/(19 − 19*X) |
| acids | | | | |
| Triglycerides | (19 − 19*X)/(20 + 20*X) | (19 + 19*X)/(20 − 20*X) | 1 | |
| MUFAs (total) | (15 − 15*X)/(20 + 20*X) | (15 + 15*X)/(20 − 20*X) | (15 − 15*X)/(19 + 19*X) | (15 + 15*X)/(19 − 19*X) |
| LPCs (total) | (7 − 7*X)/(20 + 20*X) | (7 + 7*X)/(20 − 20*X) | (7 − 7*X)/(19 + 19*X) | (7 + 7*X)/(19 − 19*X) |
| Linoleic acid | (6 − 6*X)/(20 + 20*X) | (6 + 6*X)/(20 − 20*X) | (6 − 6*X)/(19 + 19*X) | (6 + 6*X)/(19 − 19*X) |
| DHA | (4 − 4*X)/(20 + 20*X) | (4 + 4*X)/(20 − 20*X) | (4 − 4*X)/(19 + 19*X) | (4 + 4*X)/(19 − 19*X) |
| Oleic acid | (4 − 4*X)/(20 + 20*X) | (4 + 4*X)/(20 − 20*X) | (4 − 4*X)/(19 + 19*X) | (4 + 4*X)/(19 − 19*X) |
| Choline | (4 − 4*X)/(20 + 20*X) | (4 + 4*X)/(20 − 20*X) | (4 − 4*X)/(19 + 19*X) | (4 + 4*X)/(19 − 19*X) |
| 3-hydroxybutyrate | (3 − 3*X)/(20 + 20*X) | (3 + 3*X)/(20 − 20*X) | (3 − 3*X)/(19 + 19*X) | (3 + 3*X)/(19 − 19*X) |
| Propionylcarnitine | (3 − 3*X)/(20 + 20*X) | (3 + 3*X)/(20 − 20*X) | (3 − 3*X)/(19 + 19*X) | (3 + 3*X)/(19 − 19*X) |
| Adiponectin | (2 − 2*X)/(20 + 20*X) | (2 + 2*X)/(20 − 20*X) | (2 − 2*X)/(19 + 19*X) | (2 + 2*X)/(19 − 19*X) |
| Leptin | (2 − 2*X)/(20 + 20*X) | (2 + 2*X)/(20 − 20*X) | (2 − 2*X)/(19 + 19*X) | (2 + 2*X)/(19 − 19*X) |

| | SIM(cluster)ₙ/SIM(cluster)_{MUFAs (total)} | | SIM(cluster)ₙ/SIM(cluster)_{LPCs (total)} | |
|---|---|---|---|---|
| n | Lower limit | Upper limit | Lower limit | Upper limit |
| LDL-cholesterol | (25 − 25*X)/(15 + 15*X) | (25 + 25*X)/(15 − 15*X) | (25 − 25*X)/(7 + 7*X) | (25 + 25*X)/(7 − 7*X) |
| Total cholesterol | (24 − 24*X)/(15 + 15*X) | (24 + 24*X)/(15 − 15*X) | (24 − 24*X)/(7 + 7*X) | (24 + 24*X)/(7 − 7*X) |
| PUFAs (total) | (23 − 23*X)/(15 + 15*X) | (23 + 23*X)/(15 − 15*X) | (23 − 23*X)/(7 + 7*X) | (23 + 23*X)/(7 − 7*X) |
| HDL-cholesterol | (21 − 21*X)/(15 + 15*X) | (21 + 21*X)/(15 − 15*X) | (21 − 21*X)/(7 + 7*X) | (21 + 21*X)/(7 − 7*X) |
| Saturated fatty | (20 − 20*X)/(15 + 15*X) | (20 + 20*X)/(15 − 15*X) | (20 − 20*X)/(7 + 7*X) | (20 + 20*X)/(7 − 7*X) |
| acids | | | | |
| Triglycerides | (19 − 19*X)/(15 + 15*X) | (19 + 19*X)/(15 − 15*X) | (19 − 19*X)/(7 + 7*X) | (19 + 19*X)/(7 − 7*X) |
| MUFAs (total) | 1 | | (15 − 15*X)/(7 + 7*X) | (15 + 15*X)/(7 − 7*X) |
| LPCs (total) | (7 − 7*X)/(15 + 15*X) | (7 + 7*X)/(15 − 15*X) | 1 | |
| Linoleic acid | (6 − 6*X)/(15 + 15*X) | (6 + 6*X)/(15 − 15*X) | (6 − 6*X)/(7 + 7*X) | (6 + 6*X)/(7 − 7*X) |
| DHA | (4 − 4*X)/(15 + 15*X) | (4 + 4*X)/(15 − 15*X) | (4 − 4*X)/(7 + 7*X) | (4 + 4*X)/(7 − 7*X) |
| Oleic acid | (4 − 4*X)/(15 + 15*X) | (4 + 4*X)/(15 − 15*X) | (4 − 4*X)/(7 + 7*X) | (4 + 4*X)/(7 − 7*X) |
| Choline | (4 − 4*X)/(15 + 15*X) | (4 + 4*X)/(15 − 15*X) | (4 − 4*X)/(7 + 7*X) | (4 + 4*X)/(7 − 7*X) |
| 3-hydroxybutyrate | (3 − 3*X)/(15 + 15*X) | (3 + 3*X)/(15 − 15*X) | (3 − 3*X)/(7 + 7*X) | (3 + 3*X)/(7 − 7*X) |
| Propionylcarnitine | (3 − 3*X)/(15 + 15*X) | (3 + 3*X)/(15 − 15*X) | (3 − 3*X)/(7 + 7*X) | (3 + 3*X)/(7 − 7*X) |
| Adiponectin | (2 − 2*X)/(15 + 15*X) | (2 + 2*X)/(15 − 15*X) | (2 − 2*X)/(7 + 7*X) | (2 + 2*X)/(7 − 7*X) |
| Leptin | (2 − 2*X)/(15 + 15*X) | (2 + 2*X)/(15 − 15*X) | (2 − 2*X)/(7 + 7*X) | (2 + 2*X)/(7 − 7*X) |

| | SIM(cluster)ₙ/SIM(cluster)_{Linoleic acid} | | SIM(cluster)ₙ/SIM(cluster)_{DHA} | |
|---|---|---|---|---|
| n | Lower limit | Upper limit | Lower limit | Upper limit |
| LDL-cholesterol | (25 − 25*X)/(6 + 6*X) | (25 + 25*X)/(6 − 6*X) | (25 − 25*X)/(4 + 4*X) | (25 + 25*X)/(4 − 4*X) |
| Total cholesterol | (24 − 24*X)/(6 + 6*X) | (24 + 24*X)/(6 − 6*X) | (24 − 24*X)/(4 + 4*X) | (24 + 24*X)/(4 − 4*X) |
| PUFAs (total) | (23 − 23*X)/(6 + 6*X) | (23 + 23*X)/(6 − 6*X) | (23 − 23*X)/(4 + 4*X) | (23 + 23*X)/(4 − 4*X) |
| HDL-cholesterol | (21 − 21*X)/(6 + 6*X) | (21 + 21*X)/(6 − 6*X) | (21 − 21*X)/(4 + 4*X) | (21 + 21*X)/(4 − 4*X) |
| Saturated fatty | (20 − 20*X)/(6 + 6*X) | (20 + 20*X)/(6 − 6*X) | (20 − 20*X)/(4 + 4*X) | (20 + 20*X)/(4 − 4*X) |
| acids | | | | |
| Triglycerides | (19 − 19*X)/(6 + 6*X) | (19 + 19*X)/(6 − 6*X) | (19 − 19*X)/(4 + 4*X) | (19 + 19*X)/(4 − 4*X) |
| MUFAs (total) | (15 − 15*X)/(6 + 6*X) | (15 + 15*X)/(6 − 6*X) | (15 − 15*X)/(4 + 4*X) | (15 + 15*X)/(4 − 4*X) |
| LPCs (total) | (7 − 7*X)/(6 + 6*X) | (7 + 7*X)/(6 − 6*X) | (7 − 7*X)/(4 + 4*X) | (7 + 7*X)/(4 − 4*X) |
| Linoleic acid | 1 | | (6 − 6*X)/(4 + 4*X) | (6 + 6*X)/(4 − 4*X) |
| DHA | (4 − 4*X)/(6 + 6*X) | (4 + 4*X)/(6 − 6*X) | 1 | |
| Oleic acid | (4 − 4*X)/(6 + 6*X) | (4 + 4*X)/(6 − 6*X) | (4 − 4*X)/(4 + 4*X) | (4 + 4*X)/(4 − 4*X) |
| Choline | (4 − 4*X)/(6 + 6*X) | (4 + 4*X)/(6 − 6*X) | (4 − 4*X)/(4 + 4*X) | (4 + 4*X)/(4 − 4*X) |
| 3-hydroxybutyrate | (3 − 3*X)/(6 + 6*X) | (3 + 3*X)/(6 − 6*X) | (3 − 3*X)/(4 + 4*X) | (3 + 3*X)/(4 − 4*X) |
| Propionylcarnitine | (3 − 3*X)/(6 + 6*X) | (3 + 3*X)/(6 − 6*X) | (3 − 3*X)/(4 + 4*X) | (3 + 3*X)/(4 − 4*X) |
| Adiponectin | (2 − 2*X)/(6 + 6*X) | (2 + 2*X)/(6 − 6*X) | (2 − 2*X)/(4 + 4*X) | (2 + 2*X)/(4 − 4*X) |
| Leptin | (2 − 2*X)/(6 + 6*X) | (2 + 2*X)/(6 − 6*X) | (2 − 2*X)/(4 + 4*X) | (2 + 2*X)/(4 − 4*X) |
| | SIM(cluster)ₙ/SIM(cluster)_{Oleic acid} | | SIM(cluster)ₙ/SIM(cluster)_{Choline} | |
| n | Lower limit | Upper limit | Lower limit | Upper limit |
| LDL-cholesterol | (25 − 25*X)/(4 + 4*X) | (25 + 25*X)/(4 − 4*X) | (25 − 25*X)/(4 + 4*X) | (25 + 25*X)/(4 − 4*X) |
| Total cholesterol | (24 − 24*X)/(4 + 4*X) | (24 + 24*X)/(4 − 4*X) | (24 − 24*X)/(4 + 4*X) | (24 + 24*X)/(4 − 4*X) |
| PUFAs (total) | (23 − 23*X)/(4 + 4*X) | (23 + 23*X)/(4 − 4*X) | (23 − 23*X)/(4 + 4*X) | (23 + 23*X)/(4 − 4*X) |
| HDL-cholesterol | (21 − 21*X)/(4 + 4*X) | (21 + 21*X)/(4 − 4*X) | (21 − 21*X)/(4 + 4*X) | (21 + 21*X)/(4 − 4*X) |
| Saturated fatty | (20 − 20*X)/(4 + 4*X) | (20 + 20*X)/(4 − 4*X) | (20 − 20*X)/(4 + 4*X) | (20 + 20*X)/(4 − 4*X) |
| acids | | | | |
| Triglycerides | (19 − 19*X)/(4 + 4*X) | (19 + 19*X)/(4 − 4*X) | (19 − 19*X)/(4 + 4*X) | (19 + 19*X)/(4 − 4*X) |
| MUFAs (total) | (15 − 15*X)/(4 + 4*X) | (15 + 15*X)/(4 − 4*X) | (15 − 15*X)/(4 + 4*X) | (15 + 15*X)/(4 − 4*X) |
| LPCs (total) | (7 − 7*X)/(4 + 4*X) | (7 + 7*X)/(4 − 4*X) | (7 − 7*X)/(4 + 4*X) | (7 + 7*X)/(4 − 4*X) |
| Linoleic acid | (6 − 6*X)/(4 + 4*X) | (6 + 6*X)/(4 − 4*X) | (6 − 6*X)/(4 + 4*X) | (6 + 6*X)/(4 − 4*X) |
| DHA | (4 − 4*X)/(4 + 4*X) | (4 + 4*X)/(4 − 4*X) | (4 − 4*X)/(4 + 4*X) | (4 + 4*X)/(4 − 4*X) |
| Oleic acid | 1 | | (4 − 4*X)/(4 + 4*X) | (4 + 4*X)/(4 − 4*X) |
| Choline | (4 − 4*X)/(4 + 4*X) | (4 + 4*X)/(4 − 4*X) | 1 | |
| 3-hydroxybutyrate | (3 − 3*X)/(4 + 4*X) | (3 + 3*X)/(4 − 4*X) | (3 − 3*X)/(4 + 4*X) | (3 + 3*X)/(4 − 4*X) |
| Propionylcarnitine | (3 − 3*X)/(4 + 4*X) | (3 + 3*X)/(4 − 4*X) | (3 − 3*X)/(4 + 4*X) | (3 + 3*X)/(4 − 4*X) |
| Adiponectin | (2 − 2*X)/(4 + 4*X) | (2 + 2*X)/(4 − 4*X) | (2 − 2*X)/(4 + 4*X) | (2 + 2*X)/(4 − 4*X) |
| Leptin | (2 − 2*X)/(4 + 4*X) | (2 + 2*X)/(4 − 4*X) | (2 − 2*X)/(4 + 4*X) | (2 + 2*X)/(4 − 4*X) |
| | SIM(cluster)ₙ/SIM(cluster)_{3-hydroxybutyrate} | | SIM(cluster)ₙ/SIM(cluster)_{Propionylcarnitine} | |
| n | Lower limit | Upper limit | Lower limit | Upper limit |
| LDL-cholesterol | (25 − 25*X)/(3 + 3*X) | (25 + 25*X)/(3 − 3*X) | (25 − 25*X)/(3 + 3*X) | (25 + 25*X)/(3 − 3*X) |
| Total cholesterol | (24 − 24*X)/(3 + 3*X) | (24 + 24*X)/(3 − 3*X) | (24 − 24*X)/(3 + 3*X) | (24 + 24*X)/(3 − 3*X) |
| PUFAs (total) | (23 − 23*X)/(3 + 3*X) | (23 + 23*X)/(3 − 3*X) | (23 − 23*X)/(3 + 3*X) | (23 + 23*X)/(3 − 3*X) |
| HDL-cholesterol | (21 − 21*X)/(3 + 3*X) | (21 + 21*X)/(3 − 3*X) | (21 − 21*X)/(3 + 3*X) | (21 + 21*X)/(3 − 3*X) |
| Saturated fatty | (20 − 20*X)/(3 + 3*X) | (20 + 20*X)/(3 − 3*X) | (20 − 20*X)/(3 + 3*X) | (20 + 20*X)/(3 − 3*X) |
| acids | | | | |
| Triglycerides | (19 − 19*X)/(3 + 3*X) | (19 + 19*X)/(3 − 3*X) | (19 − 19*X)/(3 + 3*X) | (19 + 19*X)/(3 − 3*X) |
| MUFAs (total) | (15 − 15*X)/(3 + 3*X) | (15 + 15*X)/(3 − 3*X) | (15 − 15*X)/(3 + 3*X) | (15 + 15*X)/(3 − 3*X) |
| LPCs (total) | (7 − 7*X)/(3 + 3*X) | (7 + 7*X)/(3 − 3*X) | (7 − 7*X)/(3 + 3*X) | (7 + 7*X)/(3 − 3*X) |
| Linoleic acid | (6 − 6*X)/(3 + 3*X) | (6 + 6*X)/(3 − 3*X) | (6 − 6*X)/(3 + 3*X) | (6 + 6*X)/(3 − 3*X) |
| DHA | (4 − 4*X)/(3 + 3*X) | (4 + 4*X)/(3 − 3*X) | (4 − 4*X)/(3 + 3*X) | (4 + 4*X)/(3 − 3*X) |
| Oleic acid | (4 − 4*X)/(3 + 3*X) | (4 + 4*X)/(3 − 3*X) | (4 − 4*X)/(3 + 3*X) | (4 + 4*X)/(3 − 3*X) |
| Choline | (4 − 4*X)/(3 + 3*X) | (4 + 4*X)/(3 − 3*X) | (4 − 4*X)/(3 + 3*X) | (4 + 4*X)/(3 − 3*X) |
| 3-hydroxybutyrate | 1 | | (3 − 3*X)/(3 + 3*X) | (3 + 3*X)/(3 − 3*X) |
| Propionylcarnitine | (3 − 3*X)/(3 + 3*X) | (3 + 3*X)/(3 − 3*X) | 1 | |
| Adiponectin | (2 − 2*X)/(3 + 3*X) | (2 + 2*X)/(3 − 3*X) | (2 − 2*X)/(3 + 3*X) | (2 + 2*X)/(3 − 3*X) |
| Leptin | (2 − 2*X)/(3 + 3*X) | (2 + 2*X)/(3 − 3*X) | (2 − 2*X)/(3 + 3*X) | (2 + 2*X)/(3 − 3*X) |
| | SIM(cluster)ₙ/SIM(cluster)_{Adiponectin} | | SIM(cluster)ₙ/SIM(cluster)_{Leptin} | |
| n | Lower limit | Upper limit | Lower limit | Upper limit |
| LDL-cholesterol | (25 − 25*X)/(2 + 2*X) | (25 + 25*X)/(2 − 2*X) | (25 − 25*X)/(2 + 2*X) | (25 + 25*X)/(2 − 2*X) |
| Total cholesterol | (24 − 24*X)/(2 + 2*X) | (24 + 24*X)/(2 − 2*X) | (24 − 24*X)/(2 + 2*X) | (24 + 24*X)/(2 − 2*X) |
| PUFAs (total) | (23 − 23*X)/(2 + 2*X) | (23 + 23*X)/(2 − 2*X) | (23 − 23*X)/(2 + 2*X) | (23 + 23*X)/(2 − 2*X) |
| HDL-cholesterol | (21 − 21*X)/(2 + 2*X) | (21 + 21*X)/(2 − 2*X) | (21 − 21*X)/(2 + 2*X) | (21 + 21*X)/(2 − 2*X) |
| Saturated fatty | (20 − 20*X)/(2 + 2*X) | (20 + 20*X)/(2 − 2*X) | (20 − 20*X)/(2 + 2*X) | (20 + 20*X)/(2 − 2*X) |
| acids | | | | |
| Triglycerides | (19 − 19*X)/(2 + 2*X) | (19 + 19*X)/(2 − 2*X) | (19 − 19*X)/(2 + 2*X) | (19 + 19*X)/(2 − 2*X) |
| MUFAs (total) | (15 − 15*X)/(2 + 2*X) | (15 + 15*X)/(2 − 2*X) | (15 − 15*X)/(2 + 2*X) | (15 + 15*X)/(2 − 2*X) |
| LPCs (total) | (7 − 7*X)/(2 + 2*X) | (7 + 7*X)/(2 − 2*X) | (7 − 7*X)/(2 + 2*X) | (7 + 7*X)/(2 − 2*X) |
| Linoleic acid | (6 − 6*X)/(2 + 2*X) | (6 + 6*X)/(2 − 2*X) | (6 − 6*X)/(2 + 2*X) | (6 + 6*X)/(2 − 2*X) |
| DHA | (4 − 4*X)/(2 + 2*X) | (4 + 4*X)/(2 − 2*X) | (4 − 4*X)/(2 + 2*X) | (4 + 4*X)/(2 − 2*X) |
| Oleic acid | (4 − 4*X)/(2 + 2*X) | (4 + 4*X)/(2 − 2*X) | (4 − 4*X)/(2 + 2*X) | (4 + 4*X)/(2 − 2*X) |
| Choline | (4 − 4*X)/(2 + 2*X) | (4 + 4*X)/(2 − 2*X) | (4 − 4*X)/(2 + 2*X) | (4 + 4*X)/(2 − 2*X) |
| 3-hydroxybutyrate | (3 − 3*X)/(2 + 2*X) | (3 + 3*X)/(2 − 2*X) | (3 − 3*X)/(2 + 2*X) | (3 + 3*X)/(2 − 2*X) |
| Propionylcarnitine | (3 − 3*X)/(2 + 2*X) | (3 + 3*X)/(2 − 2*X) | (3 − 3*X)/(2 + 2*X) | (3 + 3*X)/(2 − 2*X) |

| | | | |
|---|---|---|---|
| Adiponectin | 1 | (2 − 2*X)/(2 + 2*X) | (2 + 2*X)/(2 − 2*X) |

| | | | |
|---|---|---|---|
| Leptin | (2 − 2*X)/(2 + 2*X) | (2 + 2*X)/(2 − 2*X) | 1 |

Wherein “SIM(cluster)” is the Score of individual marker (punctuation) of a particular marker determined in the isolated sample for a particular cluster; “n” is one specific biomarker of the cluster; and “cluster” is lipid metabolism cluster.

TABLE H
Inflammation cluster: ranges for the proportions between biomarkers' SIM values.

| | SIM(cluster)ₙ/SIM(cluster)_{CRP} | | SIM(cluster)ₙ/SIM(cluster)_{N-acetylglycoproteins} | |
|---|---|---|---|---|
| n | Lower limit | Upper limit | Lower limit | Upper limit |
| CRP | 1 | | (25 − 25*X)/(25 + 25*X) | (25 + 25*X)/(25 − 25*X) |
| N-acetylglycoproteins | (25 − 25*X)/(25 + 25*X) | (25 + 25*X)/(25 − 25*X) | 1 | |
| MCP 1 | (25 − 25*X)/(25 + 25*X) | (25 + 25*X)/(25 − 25*X) | (25 − 25*X)/(25 + 25*X) | (25 + 25*X)/(25 − 25*X) |
| TNFα | (23 − 23*X)/(25 + 25*X) | (23 + 23*X)/(25 − 25*X) | (23 − 23*X)/(25 + 25*X) | (23 + 23*X)/(25 − 25*X) |
| IL-6 | (20 − 20*X)/(25 + 25*X) | (20 + 20*X)/(25 − 25*X) | (20 − 20*X)/(25 + 25*X) | (20 + 20*X)/(25 − 25*X) |
| IL-10 | (18 − 18*X)/(25 + 25*X) | (18 + 18*X)/(25 − 25*X) | (18 − 18*X)/(25 + 25*X) | (18 + 18*X)/(25 − 25*X) |
| SFAs | (17 − 17*X)/(25 + 25*X) | (17 + 17*X)/(25 − 25*X) | (17 − 17*X)/(25 + 25*X) | (17 + 17*X)/(25 − 25*X) |
| sICAM1 | (15 − 15*X)/(25 + 25*X) | (15 + 15*X)/(25 − 25*X) | (15 − 15*X)/(25 + 25*X) | (15 + 15*X)/(25 − 25*X) |
| LPCs (total) | (13 − 13*X)/(25 + 25*X) | (13 + 13*X)/(25 − 25*X) | (13 − 13*X)/(25 + 25*X) | (13 + 13*X)/(25 − 25*X) |
| LBP | (13 − 13*X)/(25 + 25*X) | (13 + 13*X)/(25 − 25*X) | (13 − 13*X)/(25 + 25*X) | (13 + 13*X)/(25 − 25*X) |
| DHA | (10 − 10*X)/(25 + 25*X) | (10 + 10*X)/(25 − 25*X) | (10 − 10*X)/(25 + 25*X) | (10 + 10*X)/(25 − 25*X) |
| sCD14 | (10 − 10*X)/(25 + 25*X) | (10 + 10*X)/(25 − 25*X) | (10 − 10*X)/(25 + 25*X) | (10 + 10*X)/(25 − 25*X) |
| Linoleic acid | (7 − 7*X)/(25 + 25*X) | (7 + 7*X)/(25 − 25*X) | (7 − 7*X)/(25 + 25*X) | (7 + 7*X)/(25 − 25*X) |
| PUFAs | (5 − 5*X)/(25 + 25*X) | (5 + 5*X)/(25 − 25*X) | (5 − 5*X)/(25 + 25*X) | (5 + 5*X)/(25 − 25*X) |
| | SIM(cluster)ₙ/SIM(cluster)_{MCP 1} | | SIM(cluster)ₙ/SIM(cluster)_{TNFα} | |
| n | Lower limit | Upper limit | Lower limit | Upper limit |
| CRP | (25 − 25*X)/(25 + 25*X) | (25 + 25*X)/(25 − 25*X) | (25 − 25*X)/(23 + 23*X) | (25 + 25*X)/(23 − 23*X) |
| N-acetylglycoproteins | (25 − 25*X)/(25 + 25*X) | (25 + 25*X)/(25 − 25*X) | (25 − 25*X)/(23 + 23*X) | (25 + 25*X)/(23 − 23*X) |
| MCP 1 | 1 | | (25 − 25*X)/(23 + 23*X) | (25 + 25*X)/(23 − 23*X) |
| TNFα | (23 − 23*X)/(25 + 25*X) | (23 + 23*X)/(25 − 25*X) | 1 | |
| IL-6 | (20 − 20*X)/(25 + 25*X) | (20 + 20*X)/(25 − 25*X) | (20 − 20*X)/(23 + 23*X) | (20 + 20*X)/(23 − 23*X) |
| IL-10 | (18 − 18*X)/(25 + 25*X) | (18 + 18*X)/(25 − 25*X) | (18 − 18*X)/(23 + 23*X) | (18 + 18*X)/(23 − 23*X) |
| SFAs | (17 − 17*X)/(25 + 25*X) | (17 + 17*X)/(25 − 25*X) | (17 − 17*X)/(23 + 23*X) | (17 + 17*X)/(23 − 23*X) |
| sICAM1 | (15 − 15*X)/(25 + 25*X) | (15 + 15*X)/(25 − 25*X) | (15 − 15*X)/(23 + 23*X) | (15 + 15*X)/(23 − 23*X) |
| LPCs (total) | (13 − 13*X)/(25 + 25*X) | (13 + 13*X)/(25 − 25*X) | (13 − 13*X)/(23 + 23*X) | (13 + 13*X)/(23 − 23*X) |
| LBP | (13 − 13*X)/(25 + 25*X) | (13 + 13*X)/(25 − 25*X) | (13 − 13*X)/(23 + 23*X) | (13 + 13*X)/(23 − 23*X) |
| DHA | (10 − 10*X)/(25 + 25*X) | (10 + 10*X)/(25 − 25*X) | (10 − 10*X)/(23 + 23*X) | (10 + 10*X)/(23 − 23*X) |
| sCD14 | (10 − 10*X)/(25 + 25*X) | (10 + 10*X)/(25 − 25*X) | (10 − 10*X)/(23 + 23*X) | (10 + 10*X)/(23 − 23*X) |
| Linoleic acid | (7 − 7*X)/(25 + 25*X) | (7 + 7*X)/(25 − 25*X) | (7 − 7*X)/(23 + 23*X) | (7 + 7*X)/(23 − 23*X) |
| PUFAs | (5 − 5*X)/(25 + 25*X) | (5 + 5*X)/(25 − 25*X) | (5 − 5*X)/(23 + 23*X) | (5 + 5*X)/(23 − 23*X) |
| | SIM(cluster)ₙ/SIM(cluster)_{IL-6} | | SIM(cluster)ₙ/SIM(cluster)_{IL-10} | |
| n | Lower limit | Upper limit | Lower limit | Upper limit |
| CRP | (25 − 25*X)/(20 + 20*X) | (25 + 25*X)/(20 − 20*X) | (25 − 25*X)/(18 + 18*X) | (25 + 25*X)/(18 − 18*X) |
| N-acetylglycoproteins | (25 − 25*X)/(20 + 20*X) | (25 + 25*X)/(20 − 20*X) | (25 − 25*X)/(18 + 18*X) | (25 + 25*X)/(18 − 18*X) |
| MCP 1 | (25 − 25*X)/(20 + 20*X) | (25 + 25*X)/(20 − 20*X) | (25 − 25*X)/(18 + 18*X) | (25 + 25*X)/(18 − 18*X) |
| TNFα | (23 − 23*X)/(20 + 20*X) | (23 + 23*X)/(20 − 20*X) | (23 − 23*X)/(18 + 18*X) | (23 + 23*X)/(18 − 18*X) |
| IL-6 | 1 | | (20 − 20*X)/(18 + 18*X) | (20 + 20*X)/(18 − 18*X) |
| IL-10 | (18 − 18*X)/(20 + 20*X) | (18 + 18*X)/(20 − 20*X) | 1 | |
| SFAs | (17 − 17*X)/(20 + 20*X) | (17 + 17*X)/(20 − 20*X) | (17 − 17*X)/(18 + 18*X) | (17 + 17*X)/(18 − 18*X) |
| sICAM1 | (15 − 15*X)/(20 + 20*X) | (15 + 15*X)/(20 − 20*X) | (15 − 15*X)/(18 + 18*X) | (15 + 15*X)/(18 − 18*X) |
| LPCs (total) | (13 − 13*X)/(20 + 20*X) | (13 + 13*X)/(20 − 20*X) | (13 − 13*X)/(18 + 18*X) | (13 + 13*X)/(18 − 18*X) |
| LBP | (13 − 13*X)/(20 + 20*X) | (13 + 13*X)/(20 − 20*X) | (13 − 13*X)/(18 + 18*X) | (13 + 13*X)/(18 − 18*X) |
| DHA | (10 − 10*X)/(20 + 20*X) | (10 + 10*X)/(20 − 20*X) | (10 − 10*X)/(18 + 18*X) | (10 + 10*X)/(18 − 18*X) |
| sCD14 | (10 − 10*X)/(20 + 20*X) | (10 + 10*X)/(20 − 20*X) | (10 − 10*X)/(18 + 18*X) | (10 + 10*X)/(18 − 18*X) |
| Linoleic acid | (7 − 7*X)/(20 + 20*X) | (7 + 7*X)/(20 − 20*X) | (7 − 7*X)/(18 + 18*X) | (7 + 7*X)/(18 − 18*X) |
| PUFAs | (5 − 5*X)/(20 + 20*X) | (5 + 5*X)/(20 − 20*X) | (5 − 5*X)/(18 + 18*X) | (5 + 5*X)/(18 − 18*X) |
| | SIM(cluster)ₙ/SIM(cluster)_{SFAs} | | SIM(cluster)ₙ/SIM(cluster)_{sICAM1} | |
| n | Lower limit | Upper limit | Lower limit | Upper limit |
| CRP | (25 − 25*X)/(17 + 17*X) | (25 + 25*X)/(17 − 17*X) | (25 − 25*X)/(15 + 15*X) | (25 + 25*X)/(15 − 15*X) |
| N-acetylglycoproteins | (25 − 25*X)/(17 + 17*X) | (25 + 25*X)/(17 − 17*X | (25 − 25*X)/(15 + 15*X) | (25 + 25*X)/(15 − 15*X) |
| MCP 1 | (25 − 25*X)/(17 + 17*X) | (25 + 25*X)/(17 − 17*X) | (25 − 25*X)/(15 + 15*X) | (25 + 25*X)/(15 − 15*X) |
| TNFα | (23 − 23*X)/(17 + 17*X) | (23 + 23*X)/(17 − 17*X) | (23 − 23*X)/(15 + 15*X) | (23 + 23*X)/(15 − 15*X) |
| IL-6 | (20 − 20*X)/(17 + 17*X) | (20 + 20*X)/(17 − 17*X) | (20 − 20*X)/(15 + 15*X) | (20 + 20*X)/(15 − 15*X) |
| IL-10 | (18 − 18*X)/(17 + 17*X) | (18 + 18*X)/(17 − 17*X) | (18 − 18*X)/(15 + 15*X) | (18 + 18*X)/(15 − 15*X) |
| SFAs | 1 | | (17 − 17*X)/(15 + 15*X) | (17 + 17*X)/(15 − 15*X) |
| sICAM1 | (15 − 15*X)/(17 + 17*X) | (15 + 15*X)/(17 − 17*X) | 1 | |
| LPCs (total) | (13 − 13*X)/(17 + 17*X) | (13 + 13*X)/(17 − 17*X) | (13 − 13*X)/(15 + 15*X) | (13 + 13*X)/(15 − 15*X) |
| LBP | (13 − 13*X)/(17 + 17*X) | (13 + 13*X)/(17 − 17*X) | (13 − 13*X)/(15 + 15*X) | (13 + 13*X)/(15 − 15*X) |
| DHA | (10 − 10*X)/(17 + 17*X) | (10 + 10*X)/(17 − 17*X) | (10 − 10*X)/(15 + 15*X) | (10 + 10*X)/(15 − 15*X) |
| sCD14 | (10 − 10*X)/(17 + 17*X) | (10 + 10*X)/(17 − 17*X) | (10 − 10*X)/(15 + 15*X) | (10 + 10*X)/(15 − 15*X) |
| Linoleic acid | (7 − 7*X)/(17 + 17*X) | (7 + 7*X)/(17 − 17*X) | (7 − 7*X)/(15 + 15*X) | (7 + 7*X)/(15 − 15*X) |
| PUFAs | (5 − 5*X)/(17 + 17*X) | (5 + 5*X)/(17 − 17*X) | (5 − 5*X)/(15 + 15*X) | (5 + 5*X)/(15 − 15*X) |
| | SIM(cluster)ₙ/SIM(cluster)_{LPCs (total)} | | SIM(cluster)ₙ/SIM(cluster)_{LBP} | |
| n | Lower limit | Upper limit | Lower limit | Upper limit |
| CRP | (25 − 25*X)/(13 + 13*X) | (25 + 25*X)/(13 − 13*X) | (25 − 25*X)/(13 + 13*X) | (25 + 25*X)/(13 − 13*X) |
| N-acetylglycoproteins | (25 − 25*X)/(13 + 13*X) | (25 + 25*X)/(13 − 13*X) | (25 − 25*X)/(13 + 13*X) | (25 + 25*X)/(13 − 13*X) |
| MCP 1 | (25 − 25*X)/(13 + 13*X) | (25 + 25*X)/(13 − 13*X) | (25 − 25*X)/(13 + 13*X) | (25 + 25*X)/(13 − 13*X) |
| TNFα | (23 − 23*X)/(13 + 13*X) | (23 + 23*X)/(13 − 13*X) | (23 − 23*X)/(13 + 13*X) | (23 + 23*X)/(13 − 13*X) |
| IL-6 | (20 − 20*X)/(13 + 13*X) | (20 + 20*X)/(13 − 13*X) | (20 − 20*X)/(13 + 13*X) | (20 + 20*X)/(13 − 13*X) |
| IL-10 | (18 − 18*X)/(13 + 13*X) | (18 + 18*X)/(13 − 13*X) | (18 − 18*X)/(13 + 13*X) | (18 + 18*X)/(13 − 13*X) |
| SFAs | (17 − 17*X)/(13 + 13*X) | (17 + 17*X)/(13 − 13*X) | (17 − 17*X)/(13 + 13*X) | (17 + 17*X)/(13 − 13*X) |
| sICAM1 | (15 − 15*X)/(13 + 13*X) | (15 + 15*X)/(13 − 13*X) | (15 − 15*X)/(13 + 13*X) | (15 + 15*X)/(13 − 13*X) |
| LPCs (total) | 1 | | (13 − 13*X)/(13 + 13*X) | (13 + 13*X)/(13 − 13*X) |
| LBP | (13 − 13*X)/(13 + 13*X) | (13 + 13*X)/(13 − 13*X) | 1 | |
| DHA | (10 − 10*X)/(13 + 13*X) | (10 + 10*X)/(13 − 13*X) | (10 − 10*X)/(13 + 13*X) | (10 + 10*X)/(13 − 13*X) |
| sCD14 | (10 − 10*X)/(13 + 13*X) | (10 + 10*X)/(13 − 13*X) | (10 − 10*X)/(13 + 13*X) | (10 + 10*X)/(13 − 13*X) |
| Linoleic acid | (7 − 7*X)/(13 + 13*X) | (7 + 7*X)/(13 − 13*X) | (7 − 7*X)/(13 + 13*X) | (7 + 7*X)/(13 − 13*X) |
| PUFAs | (5 − 5*X)/(13 + 13*X) | (5 + 5*X)/(13 − 13*X) | (5 − 5*X)/(13 + 13*X) | (5 + 5*X)/(13 − 13*X) |
| | SIM(cluster)ₙ/SIM(cluster)_{DHA} | | SIM(cluster)ₙ/SIM(cluster)_{sCD14} | |
| n | Lower limit | Upper limit | Lower limit | Upper limit |
| CRP | (25 − 25*X)/(10 + 10*X) | (25 + 25*X)/(10 − 10*X) | (25 − 25*X)/(10 + 10*X) | (25 + 25*X)/(10 − 10*X) |
| N-acetylglycoproteins | (25 − 25*X)/(10 + 10*X) | (25 + 25*X)/(10 − 10*X) | (25 − 25*X)/(10 + 10*X) | (25 + 25*X)/(10 − 10*X) |
| MCP 1 | (25 − 25*X)/(10 + 10*X) | (25 + 25*X)/(10 − 10*X) | (25 − 25*X)/(10 + 10*X) | (25 + 25*X)/(10 − 10*X) |
| TNFα | (23 − 23*X)/(10 + 10*X) | (23 + 23*X)/(10 − 10*X) | (23 − 23*X)/(10 + 10*X) | (23 + 23*X)/(10 − 10*X) |
| IL-6 | (20 − 20*X)/(10 + 10*X) | (20 + 20*X)/(10 − 10*X) | (20 − 20*X)/(10 + 10*X) | (20 + 20*X)/(10 − 10*X) |
| IL-10 | (18 − 18*X)/(10 + 10*X) | (18 + 18*X)/(10 − 10*X) | (18 − 18*X)/(10 + 10*X) | (18 + 18*X)/(10 − 10*X) |
| SFAs | (17 − 17*X)/(10 + 10*X) | (17 + 17*X)/(10 − 10*X) | (17 − 17*X)/(10 + 10*X) | (17 + 17*X)/(10 − 10*X) |
| sICAM1 | (15 − 15*X)/(10 + 10*X) | (15 + 15*X)/(10 − 10*X) | (15 − 15*X)/(10 + 10*X) | (15 + 15*X)/(10 − 10*X) |
| LPCs (total) | (13 − 13*X)/(10 + 10*X) | (13 + 13*X)/(10 − 10*X) | (13 − 13*X)/(10 + 10*X) | (13 + 13*X)/(10 − 10*X) |
| LBP | (13 − 13*X)/(10 + 10*X) | (13 + 13*X)/(10 − 10*X) | (13 − 13*X)/(10 + 10*X) | (13 + 13*X)/(10 − 10*X) |
| DHA | 1 | | (10 − 10*X)/(10 + 10*X) | (10 + 10*X)/(10 − 10*X) |
| sCD14 | (10 − 10*X)/(10 + 10*X) | (10 + 10*X)/(10 − 10*X) | 1 | |
| Linoleic acid | (7 − 7*X)/(10 + 10*X) | (7 + 7*X)/(10 − 10*X) | (7 − 7*X)/(10 + 10*X) | (7 + 7*X)/(10 − 10*X) |
| PUFAs | (5 − 5*X)/(10 + 10*X) | (5 + 5*X)/(10 − 10*X) | (5 − 5*X)/(10 + 10*X) | (5 + 5*X)/(10 − 10*X) |
| | SIM(cluster)ₙ/SIM(cluster)_{Linoleic acid} | | SIM(cluster)ₙ/SIM(cluster)_{PUFAs} | |
| n | Lower limit | Upper limit | Lower limit | Upper limit |
| CRP | (25 − 25*X)/(7 + 7*X) | (25 + 25*X)/(7 − 7*X) | (25 − 25*X)/(5 + 5*X) | (25 + 25*X)/(5 − 5*X) |
| N-acetylglycoproteins | (25 − 25*X)/(7 + 7*X) | (25 + 25*X)/(7 − 7*X) | (25 − 25*X)/(5 + 5*X) | (25 + 25*X)/(5 − 5*X) |
| MCP 1 | (25 − 25*X)/(7 + 7*X) | (25 + 25*X)/(7 − 7*X) | (25 − 25*X)/(5 + 5*X) | (25 + 25*X)/(5 − 5*X) |
| TNFα | (23 − 23*X)/(7 + 7*X) | (23 + 23*X)/(7 − 7*X) | (23 − 23*X)/(5 + 5*X) | (23 + 23*X)/(5 − 5*X) |
| IL-6 | (20 − 20*X)/(7 + 7*X) | (20 + 20*X)/(7 − 7*X) | (20 − 20*X)/(5 + 5*X) | (20 + 20*X)/(5 − 5*X) |
| IL-10 | (18 − 18*X)/(7 + 7*X) | (18 + 18*X)/(7 − 7*X) | (18 − 18*X)/(5 + 5*X) | (18 + 18*X)/(5 − 5*X) |
| SFAs | (17 − 17*X)/(7 + 7*X) | (17 + 17*X)/(7 − 7*X) | (17 − 17*X)/(5 + 5*X) | (17 + 17*X)/(5 − 5*X) |
| sICAM1 | (15 − 15*X)/(7 + 7*X) | (15 + 15*X)/(7 − 7*X) | (15 − 15*X)/(5 + 5*X) | (15 + 15*X)/(5 − 5*X) |
| LPCs (total) | (13 − 13*X)/(7 + 7*X) | (13 + 13*X)/(7 − 7*X) | (13 − 13*X)/(5 + 5*X) | (13 + 13*X)/(5 − 5*X) |
| LBP | (13 − 13*X)/(7 + 7*X) | (13 + 13*X)/(7 − 7*X) | (13 − 13*X)/(5 + 5*X) | (13 + 13*X)/(5 − 5*X) |
| DHA | (10 − 10*X)/(7 + 7*X) | (10 + 10*X)/(7 − 7*X) | (10 − 10*X)/(5 + 5*X) | (10 + 10*X)/(5 − 5*X) |
| sCD14 | (10 − 10*X)/(7 + 7*X) | (10 + 10*X)/(7 − 7*X) | (10 − 10*X)/(5 + 5*X) | (10 + 10*X)/(5 − 5*X) |

| | | | |
|---|---|---|---|
| Linoleic acid | 1 | (7 − 7*X)/(5 + 5*X) | (7 + 7*X)/(5 − 5*X) |

| | | | |
|---|---|---|---|
| PUFAs | (5 − 5*X)/(7 + 7*X) | (5 + 5*X)/(7 − 7*X) | 1 |

Wherein “SIM(cluster)” is the Score of individual marker (punctuation) of a particular marker determined in the isolated sample for a particular cluster; “n” is one specific biomarker of the cluster; and “cluster” is inflammation cluster.

TABLE I
Microbiota cluster: ranges for the proportions between biomarkers' SIM values.

| | SIM(cluster)ₙ/SIM(cluster)_{TMA} | | SIM(cluster)ₙ/SIM(cluster)_{TMAO} | |
|---|---|---|---|---|
| n | Lower limit | Upper limit | Lower limit | Upper limit |
| TMA | 1 | | (25 − 25*X)/(25 + 25*X) | (25 + 25*X)/(25 − 25*X) |
| TMAO | (25 − 25*X)/(25 + 25*X) | (25 + 25*X)/(25 − 25*X) | 1 | |
| Betaine | (20 − 20*X)/(25 + 25*X) | (20 + 20*X)/(25 − 25*X) | (20 − 20*X)/(25 + 25*X) | (20 + 20*X)/(25 − 25*X) |
| Choline | (20 − 20*X)/(25 + 25*X) | (20 + 20*X)/(25 − 25*X) | (20 − 20*X)/(25 + 25*X) | (20 + 20*X)/(25 − 25*X) |
| DMA | (15 − 15*X)/(25 + 25*X) | (15 + 15*X)/(25 − 25*X) | (15 − 15*X)/(25 + 25*X) | (15 + 15*X)/(25 − 25*X) |
| Dimethylglycine | (12 − 12*X)/(25 + 25*X) | (12 + 12*X)/(25 − 25*X) | (12 − 12*X)/(25 + 25*X) | (12 + 12*X)/(25 − 25*X) |
| LBP | (10 − 10*X)/(25 + 25*X) | (10 + 10*X)/(25 − 25*X) | (10 − 10*X)/(25 + 25*X) | (10 + 10*X)/(25 − 25*X) |
| Succinate | (8 − 8*X)/(25 + 25*X) | (8 + 8*X)/(25 − 25*X) | (8 − 8*X)/(25 + 25*X) | (8 + 8*X)/(25 − 25*X) |
| Lactate | (8 − 8*X)/(25 + 25*X) | (8 + 8*X)/(25 − 25*X) | (8 − 8*X)/(25 + 25*X) | (8 + 8*X)/(25 − 25*X) |
| Acetate | (6 − 6*X)/(25 + 25*X) | (6 + 6*X)/(25 − 25*X) | (6 − 6*X)/(25 + 25*X) | (6 + 6*X)/(25 − 25*X) |
| | SIM(cluster)ₙ/SIM(cluster)_{Betaine} | | SIM(cluster)ₙ/SIM(cluster)_{Choline} | |
| n | Lower limit | Upper limit | Lower limit | Upper limit |
| TMA | (25 − 25*X)/(20 + 20*X) | (25 + 25*X)/(20 − 20*X) | (25 − 25*X)/(20 + 20*X) | (25 + 25*X)/(20 − 20*X) |
| TMAO | (25 − 25*X)/(20 + 20*X) | (25 + 25*X)/(20 − 20*X) | (25 − 25*X)/(20 + 20*X) | (25 + 25*X)/(20 − 20*X) |
| Betaine | 1 | | (20 − 20*X)/(20 + 20*X) | (20 + 20*X)/(20 − 20*X) |
| Choline | (20 − 20*X)/(20 + 20*X) | (20 + 20*X)/(20 − 20*X) | 1 | |
| DMA | (15 − 15*X)/(20 + 20*X) | (15 + 15*X)/(20 − 20*X) | (15 − 15*X)/(20 + 20*X) | (15 + 15*X)/(20 − 20*X) |
| Dimethylglycine | (12 − 12*X)/(20 + 20*X) | (12 + 12*X)/(20 − 20*X) | (12 − 12*X)/(20 + 20*X) | (12 + 12*X)/(20 − 20*X) |
| LBP | (10 − 10*X)/(20 + 20*X) | (10 + 10*X)/(20 − 20*X) | (10 − 10*X)/(20 + 20*X) | (10 + 10*X)/(20 − 20*X) |
| Succinate | (8 − 8*X)/(20 + 20*X) | (8 + 8*X)/(20 − 20*X) | (8 − 8*X)/(20 + 20*X) | (8 + 8*X)/(20 − 20*X) |
| Lactate | (8 − 8*X)/(20 + 20*X) | (8 + 8*X)/(20 − 20*X) | (8 − 8*X)/(20 + 20*X) | (8 + 8*X)/(20 − 20*X) |
| Acetate | (6 − 6*X)/(20 + 20*X) | (6 + 6*X)/(20 − 20*X) | (6 − 6*X)/(20 + 20*X) | (6 + 6*X)/(20 − 20*X) |
| | SIM(cluster)ₙ/SIM(cluster)_{DMA} | | SIM(cluster)ₙ/SIM(cluster)_{Dimethylglycine} | |
| n | Lower limit | Upper limit | Lower limit | Upper limit |
| TMA | (25 − 25*X)/(15 + 15*X) | (25 + 25*X)/(15 − 15*X) | (25 − 25*X)/(12 + 12*X) | (25 + 25*X)/(12 − 12*X) |
| TMAO | (25 − 25*X)/(15 + 15*X) | (25 + 25*X)/(15 − 15*X) | (25 − 25*X)/(12 + 12*X) | (25 + 25*X)/(12 − 12*X) |
| Betaine | (20 − 20*X)/(15 + 15*X) | (20 + 20*X)/(15 − 15*X) | (20 − 20*X)/(12 + 12*X) | (20 + 20*X)/(12 − 12*X) |
| Choline | (20 − 20*X)/(15 + 15*X) | (20 + 20*X)/(15 − 15*X) | (20 − 20*X)/(12 + 12*X) | (20 + 20*X)/(12 − 12*X) |
| DMA | 1 | | (15 − 15*X)/(12 + 12*X) | (15 + 15*X)/(12 − 12*X) |
| Dimethylglycine | (12 − 12*X)/(15 + 15*X) | (12 + 12*X)/(15 − 15*X) | 1 | |
| LBP | (10 − 10*X)/(15 + 15*X) | (10 + 10*X)/(15 − 15*X) | (10 − 10*X)/(12 + 12*X) | (10 + 10*X)/(12 − 12*X) |
| Succinate | (8 − 8*X)/(15 + 15*X) | (8 + 8*X)/(15 − 15*X) | (8 − 8*X)/(12 + 12*X) | (8 + 8*X)/(12 − 12*X) |
| Lactate | (8 − 8*X)/(15 + 15*X) | (8 + 8*X)/(15 − 15*X) | (8 − 8*X)/(12 + 12*X) | (8 + 8*X)/(12 − 12*X) |
| Acetate | (6 − 6*X)/(15 + 15*X) | (6 + 6*X)/(15 − 15*X) | (6 − 6*X)/(12 + 12*X) | (6 + 6*X)/(12 − 12*X) |

| | SIM(cluster)ₙ/SIM(cluster)_{LBP} | | SIM(cluster)ₙ/SIM(cluster)_{Succinate} | |
|---|---|---|---|---|
| n | Lower limit | Upper limit | Lower limit | Upper limit |
| TMA | (25 − 25*X)/(10 + 10*X) | (25 + 25*X)/(10 − 10*X) | (25 − 25*X)/(8 + 8*X) | (25 + 25*X)/(8 − 8*X) |
| TMAO | (25 − 25*X)/(10 + 10*X) | (25 + 25*X)/(10 − 10*X) | (25 − 25*X)/(8 + 8*X) | (25 + 25*X)/(8 − 8*X) |
| Betaine | (20 − 20*X)/(10 + 10*X) | (20 + 20*X)/(10 − 10*X) | (20 − 20*X)/(8 + 8*X) | (20 + 20*X)/(8 − 8*X) |
| Choline | (20 − 20*X)/(10 + 10*X) | (20 + 20*X)/(10 − 10*X) | (20 − 20*X)/(8 + 8*X) | (20 + 20*X)/(8 − 8*X) |
| DMA | (15 − 15*X)/(10 + 10*X) | (15 + 15*X)/(10 − 10*X) | (15 − 15*X)/(8 + 8*X) | (15 + 15*X)/(8 − 8*X) |
| Dimethylglycine | (12 − 12*X)/(10 + 10*X) | (12 + 12*X)/(10 − 10*X) | (12 − 12*X)/(8 + 8*X) | (12 + 12*X)/(8 − 8*X) |
| LBP | 1 | | (10 − 10*X)/(8 + 8*X) | (10 + 10*X)/(8 − 8*X) |
| Succinate | (8 − 8*X)/(10 + 10*X) | (8 + 8*X)/(10 − 10*X) | 1 | |
| Lactate | (8 − 8*X)/(10 + 10*X) | (8 + 8*X)/(10 − 10*X) | (8 − 8*X)/(8 + 8*X) | (8 + 8*X)/(8 − 8*X) |
| Acetate | (6 − 6*X)/(10 + 10*X) | (6 + 6*X)/(10 − 10*X) | (6 − 6*X)/(8 + 8*X) | (6 + 6*X)/(8 − 8*X) |

| | SIM(cluster)ₙ/SIM(cluster)_{Lactate} | | SIM(cluster)ₙ/SIM(cluster)_{Acetate} | |
|---|---|---|---|---|
| n | Lower limit | Upper limit | Lower limit | Upper limit |
| TMA | (25 − 25*X)/(8 + 8*X) | (25 + 25*X)/(8 − 8*X) | (25 − 25*X)/(6 + 6*X) | (25 + 25*X)/(6 − 6*X) |
| TMAO | (25 − 25*X)/(8 + 8*X) | (25 + 25*X)/(8 − 8*X) | (25 − 25*X)/(6 + 6*X) | (25 + 25*X)/(6 − 6*X) |
| Betaine | (20 − 20*X)/(8 + 8*X) | (20 + 20*X)/(8 − 8*X) | (20 − 20*X)/(6 + 6*X) | (20 + 20*X)/(6 − 6*X) |
| Choline | (20 − 20*X)/(8 + 8*X) | (20 + 20*X)/(8 − 8*X) | (20 − 20*X)/(6 + 6*X) | (20 + 20*X)/(6 − 6*X) |
| DMA | (15 − 15*X)/(8 + 8*X) | (15 + 15*X)/(8 − 8*X) | (15 − 15*X)/(6 + 6*X) | (15 + 15*X)/(6 − 6*X) |
| Dimethylglycine | (12 − 12*X)/(8 + 8*X) | (12 + 12*X)/(8 − 8*X) | (12 − 12*X)/(6 + 6*X) | (12 + 12*X)/(6 − 6*X) |
| LBP | (10 − 10*X)/(8 + 8*X) | (10 + 10*X)/(8 − 8*X) | (10 − 10*X)/(6 + 6*X) | (10 + 10*X)/(6 − 6*X) |
| Succinate | (8 − 8*X)/(8 + 8*X) | (8 + 8*X)/(8 − 8*X) | (8 − 8*X)/(6 + 6*X) | (8 + 8*X)/(6 − 6*X) |

| | | | |
|---|---|---|---|
| Lactate | 1 | (8 − 8*X)/(6 + 6*X) | (8 + 8*X)/(6 − 6*X) |

| | | | |
|---|---|---|---|
| Acetate | (6 − 6*X)/(8 + 8*X) | (6 + 6*X)/(8 − 8*X) | 1 |

Wherein “SIM(cluster)” is the Score of individual marker (punctuation) of a particular marker determined in the isolated sample for a particular cluster; “n” is one specific biomarker of the cluster; and “cluster” is microbiota metabolism cluster.

TABLE J
Oxidative stress cluster: ranges for the proportions between biomarkers' SIM values.

| | SIM(cluster)ₙ/SIM(cluster)_{8-iso-PGF2α} | | SIM(cluster)ₙ/SIM(cluster)_{8-OHdG} | |
|---|---|---|---|---|
| n | Lower limit | Upper limit | Lower limit | Upper limit |
| 8-iso-PGF2α | 1 | | (25 − 25*X)/(23 + 23*X) | (25 + 25*X)/(23 − 23*X) |
| 8-OHdG | (23 − 23*X)/(25 + 25*X) | (23 + 23*X)/(25 − 25*X) | 1 | |
| LDLox | (21 − 21*X)/(25 + 25*X) | (21 + 21*X)/(25 − 25*X) | (21 − 21*X)/(23 + 23*X) | (21 + 21*X)/(23 − 23*X) |
| Uric acid | (21 − 21*X)/(25 + 25*X) | (21 + 21*X)/(25 − 25*X) | (21 − 21*X)/(23 + 23*X) | (21 + 21*X)/(23 − 23*X) |
| Allantoin | (18 − 18*X)/(25 + 25*X) | (18 + 18*X)/(25 − 25*X) | (18 − 18*X)/(23 + 23*X) | (18 + 18*X)/(23 − 23*X) |
| Betaine | (15 − 15*X)/(25 + 25*X) | (15 + 15*X)/(25 − 25*X) | (15 − 15*X)/(23 + 23*X) | (15 + 15*X)/(23 − 23*X) |
| Pseudouridine | (13 − 13*X)/(25 + 25*X) | (13 + 13*X)/(25 − 25*X) | (13 − 13*X)/(23 + 23*X) | (13 + 13*X)/(23 − 23*X) |
| Dimethylglycine | (10 − 10*X)/(25 + 25*X) | (10 + 10*X)/(25 − 25*X) | (10 − 10*X)/(23 + 23*X) | (10 + 10*X)/(23 − 23*X) |
| Methionine | (10 − 10*X)/(25 + 25*X) | (10 + 10*X)/(25 − 25*X) | (10 − 10*X)/(23 + 23*X) | (10 + 10*X)/(23 − 23*X) |
| Glycine | (5 − 5*X)/(25 + 25*X) | (5 + 5*X)/(25 − 25*X) | (5 − 5*X)/(23 + 23*X) | (5 + 5*X)/(23 − 23*X) |
| | SIM(cluster)ₙ/SIM(cluster)_{LDLox} | | SIM(cluster)ₙ/SIM(cluster)_{Uric acid} | |
| n | Lower limit | Upper limit | Lower limit | Upper limit |
| 8-iso-PGF2α | (25 − 25*X)/(21 + 21*X) | (25 + 25*X)/(21 − 21*X) | (25 − 25*X)/(21 + 21*X) | (25 + 25*X)/(21 − 21*X) |
| 8-OHdG | (23 − 23*X)/(21 + 21*X) | (23 + 23*X)/(21 − 21*X) | (23 − 23*X)/(21 + 21*X) | (23 + 23*X)/(21 − 21*X) |
| LDLox | 1 | | (21 − 21*X)/(21 + 21*X) | (21 + 21*X)/(21 − 21*X) |
| Uric acid | (21 − 21*X)/(21 + 21*X) | (21 + 21*X)/(21 − 21*X) | 1 | |
| Allantoin | (18 − 18*X)/(21 + 21*X) | (18 + 18*X)/(21 − 21*X) | (18 − 18*X)/(21 + 21*X) | (18 + 18*X)/(21 − 21*X) |
| Betaine | (15 − 15*X)/(21 + 21*X) | (15 + 15*X)/(21 − 21*X) | (15 − 15*X)/(21 + 21*X) | (15 + 15*X)/(21 − 21*X) |
| Pseudouridine | (13 − 13*X)/(21 + 21*X) | (13 + 13*X)/(21 − 21*X) | (13 − 13*X)/(21 + 21*X) | (13 + 13*X)/(21 − 21*X) |
| Dimethylglycine | (10 − 10*X)/(21 + 21*X) | (10 + 10*X)/(21 − 21*X) | (10 − 10*X)/(21 + 21*X) | (10 + 10*X)/(21 − 21*X) |
| Methionine | (10 − 10*X)/(21 + 21*X) | (10 + 10*X)/(21 − 21*X) | (10 − 10*X)/(21 + 21*X) | (10 + 10*X)/(21 − 21*X) |
| Glycine | (5 − 5*X)/(21 + 21*X) | (5 + 5*X)/(21 − 21*X) | (5 − 5*X)/(21 + 21*X) | (5 + 5*X)/(21 − 21*X) |
| | SIM(cluster)ₙ/SIM(cluster)_{Allantoin} | | SIM(cluster)ₙ/SIM(cluster)_{Betaine} | |
| n | Lower limit | Upper limit | Lower limit | Upper limit |
| 8-iso-PGF2α | (25 − 25*X)/(18 + 18*X) | (25 + 25*X)/(18 − 18*X) | (25 − 25*X)/(15 + 15*X) | (25 + 25*X)/(15 − 15*X) |
| 8-OHdG | (23 − 23*X)/(18 + 18*X) | (23 + 23*X)/(18 − 18*X) | (23 − 23*X)/(15 + 15*X) | (23 + 23*X)/(15 − 15*X) |
| LDLox | (21 − 21*X)/(18 + 18*X) | (21 + 21*X)/(18 − 18*X) | (21 − 21*X)/(15 + 15*X) | (21 + 21*X)/(15 − 15*X) |
| Uric acid | (21 − 21*X)/(18 + 18*X) | (21 + 21*X)/(18 − 18*X) | (21 − 21*X)/(15 + 15*X) | (21 + 21*X)/(15 − 15*X) |
| Allantoin | 1 | | (18 − 18*X)/(15 + 15*X) | (18 + 18*X)/(15 − 15*X) |
| Betaine | (15 − 15*X)/(18 + 18*X) | (15 + 15*X)/(18 − 18*X) | 1 | |
| Pseudouridine | (13 − 13*X)/(18 + 18*X) | (13 + 13*X)/(18 − 18*X) | (13 − 13*X)/(15 + 15*X) | (13 + 13*X)/(15 − 15*X) |
| Dimethylglycine | (10 − 10*X)/(18 + 18*X) | (10 + 10*X)/(18 − 18*X) | (10 − 10*X)/(15 + 15*X) | (10 + 10*X)/(15 − 15*X) |
| Methionine | (10 − 10*X)/(18 + 18*X) | (10 + 10*X)/(18 − 18*X) | (10 − 10*X)/(15 + 15*X) | (10 + 10*X)/(15 − 15*X) |
| Glycine | (5 − 5*X)/(18 + 18*X) | (5 + 5*X)/(18 − 18*X) | (5 − 5*X)/(15 + 15*X) | (5 + 5*X)/(15 − 15*X) |
| | SIM(cluster)ₙ/SIM(cluster)_{Pseudouridine} | | SIM(cluster)ₙ/SIM(cluster)_{Dimethylglycine} | |
| n | Lower limit | Upper limit | Lower limit | Upper limit |
| 8-iso-PGF2α | (25 − 25*X)/(13 + 13*X) | (25 + 25*X)/(13 − 13*X) | (25 − 25*X)/(10 + 10*X) | (25 + 25*X)/(10 − 10*X) |
| 8-OHdG | (23 − 23*X)/(13 + 13*X) | (23 + 23*X)/(13 − 13*X) | (23 − 23*X)/(10 + 10*X) | (23 + 23*X)/(10 − 10*X) |
| LDLox | (21 − 21*X)/(13 + 13*X) | (21 + 21*X)/(13 − 13*X) | (21 − 21*X)/(10 + 10*X) | (21 + 21*X)/(10 − 10*X) |
| Uric acid | (21 − 21*X)/(13 + 13*X) | (21 + 21*X)/(13 − 13*X) | (21 − 21*X)/(10 + 10*X) | (21 + 21*X)/(10 − 10*X) |
| Allantoin | (18 − 18*X)/(13 + 13*X) | (18 + 18*X)/(13 − 13*X) | (18 − 18*X)/(10 + 10*X) | (18 + 18*X)/(10 − 10*X) |
| Betaine | (15 − 15*X)/(13 + 13*X) | (15 + 15*X)/(13 − 13*X) | (15 − 15*X)/(10 + 10*X) | (15 + 15*X)/(10 − 10*X) |
| Pseudouridine | 1 | | (13 − 13*X)/(10 + 10*X) | (13 + 13*X)/(10 − 10*X) |
| Dimethylglycine | (10 − 10*X)/(13 + 13*X) | (10 + 10*X)/(13 − 13*X) | 1 | |
| Methionine | (10 − 10*X)/(13 + 13*X) | (10 + 10*X)/(13 − 13*X) | (10 − 10*X)/(10 + 10*X) | (10 + 10*X)/(10 − 10*X) |
| Glycine | (5 − 5*X)/(13 + 13*X) | (5 + 5*X)/(13 − 13*X) | (5 − 5*X)/(10 + 10*X) | (5 + 5*X)/(10 − 10*X) |

| | SIM(cluster)ₙ/SIM(cluster)_{Methionine} | | SIM(cluster)ₙ/SIM(cluster)_{Glycine} | |
|---|---|---|---|---|
| n | Lower limit | Upper limit | Lower limit | Upper limit |
| 8-iso-PGF2α | (25 − 25*X)/(10 + 10*X) | (25 + 25*X)/(10 − 10*X) | (25 − 25*X)/(5 + 5*X) | (25 + 25*X)/(5 − 5*X) |
| 8-OHdG | (23 − 23*X)/(10 + 10*X) | (23 + 23*X)/(10 − 10*X) | (23 − 23*X)/(5 + 5*X) | (23 + 23*X)/(5 − 5*X) |
| LDLox | (21 − 21*X)/(10 + 10*X) | (21 + 21*X)/(10 − 10*X) | (21 − 21*X)/(5 + 5*X) | (21 + 21*X)/(5 − 5*X) |
| Uric acid | (21 − 21*X)/(10 + 10*X) | (21 + 21*X)/(10 − 10*X) | (21 − 21*X)/(5 + 5*X) | (21 + 21*X)/(5 − 5*X) |
| Allantoin | (18 − 18*X)/(10 + 10*X) | (18 + 18*X)/(10 − 10*X) | (18 − 18*X)/(5 + 5*X) | (18 + 18*X)/(5 − 5*X) |
| Betaine | (15 − 15*X)/(10 + 10*X) | (15 + 15*X)/(10 − 10*X) | (15 − 15*X)/(5 + 5*X) | (15 + 15*X)/(5 − 5*X) |
| Pseudouridine | (13 − 13*X)/(10 + 10*X) | (13 + 13*X)/(10 − 10*X) | (13 − 13*X)/(5 + 5*X) | (13 + 13*X)/(5 − 5*X) |
| Dimethylglycine | (10 − 10*X)/(10 + 10*X) | (10 + 10*X)/(10 − 10*X) | (10 − 10*X)/(5 + 5*X) | (10 + 10*X)/(5 − 5*X) |

| | | | |
|---|---|---|---|
| Methionine | 1 | (10 − 10*X)/(5 + 5*X) | (10 + 10*X)/(5 − 5*X) |

| | | | |
|---|---|---|---|
| Glycine | (5 − 5*X)/(10 + 10*X) | (5 + 5*X)/(10 − 10*X) | 1 |

Wherein “SIM(cluster)” is the Score of individual marker (punctuation) of a particular marker determined in the isolated sample for a particular cluster; “n” is one specific biomarker of the cluster; and “cluster” is oxidative stress cluster.

### EXAMPLES

### Example 1. Scoring System with Multivariant Assessment of Metabolism

49 metabolic biomarkers (i.e., markers), from the total of 51, described in Tables 1-5 below were analysed in 600 subjects (from Spain, Denmark, and from Netherlands).

The procedure to determine a personalized nutrition recommendation was based on a linear combination where the biomarkers and weights (i.e., factors) are the ones indicated in the Tables 1-5. The values of the factors were given according to the significance of each marker in the cluster. Significance was stablished by i) selecting one or more biomarkers in a cluster representative of specific metabolic processes that are biologically relevant for the cluster (i.e. carbohydrate metabolism dysregulation, lipid metabolism dysregulation, oxidative stress, inflammation and microbiota-related systemic alterations); ii) setting up models by partial least square regression (PLSR) using the rest of biomarkers to predict values of concentration of the selected biomarker iii) calculating the variable importance in the projection (VIP) score for each predictor biomarker; iv) setting factor values for each biomarker proportionally to their VIP scores in the PLSR model and also according or considering the study of the literature performed by experts in the field. Then, for each subject, the scores from the levels of the markers were calculated as indicated in formula (2).

$$Score ( cluster ) = {\sum }_{n = 1}^{Ncluster} ( {weight ( cluster )}_{n} * {biomarker ( cluster )}_{n} ) \qquad ( 2 )$$

TABLE 1
Lipid metabolism cluster
Lipid cluster

| | | | | | Normal/ |
|---|---|---|---|---|---|
| # | Biomarker | Biofluid | SIM (P) | Weight (%) | Reverse |
| 1 | LDL-cholesterol | Plasma | 25 | 13.70% | Normal |
| 2 | Total cholesterol | Plasma | 24 | 13.20% | Normal |
| 3 | PUFAs (total) | Serum | 23 | 12.60% | Reverse |
| 4 | HDL-cholesterol | Plasma | 21 | 11.50% | Reverse |
| 5 | Saturated fatty acids | Serum | 20 | 11.00% | Normal |
| 6 | Triglycerides | Plasma | 19 | 10.40% | Normal |
| 7 | MUFAs (total) | Serum | 15 | 8.20% | Normal |
| 8 | LPCs(total) | Plasma | 7 | 3.80% | Normal |
| 9 | Linoleic acid | Serum | 6 | 3.30% | Normal |
| 10 | DHA | Serum | 4 | 2.20% | Reverse |
| 10 | Oleic acid | Serum | 4 | 2.20% | Normal |
| 10 | Choline | Serum | 4 | 2.20% | Normal |
| 11 | 3-hydroxybutyrate | Serum | 3 | 1.60% | Normal |
| 11 | Propionylcarnitine | Plasma | 3 | 1.60% | Normal |
| 12 | Adiponectin | Serum | 2 | 1.10% | Reverse |
| 12 | Leptin | Serum | 2 | 1.10% | Normal |

SIM (P) stands for Score of individual marker (punctuation).

TABLE 2
Carbohydrate metabolism cluster
Carbohydrate cluster

| | | | | | Normal/ |
|---|---|---|---|---|---|
| # | Biomarker | Biofluid | SIM (P) | Weight (%) | Reverse |
| 1 | Glucose | Plasma | 25 | 11.00% | Normal |
| 1 | HOMA-IR | | 25 | 11.00% | Normal |
| 1 | Glutamate | Serum | 25 | 11.00% | Normal |
| 1 | Uric acid | Serum | 25 | 11.00% | Normal |
| 2 | Leptin | Serum | 15 | 6.60% | Normal |
| 2 | Adiponectin | Serum | 15 | 6.60% | Reverse |
| 2 | Insulin | Serum | 15 | 6.60% | Normal |
| 2 | Tyrosine | Serum | 15 | 6.60% | Normal |
| 3 | Propionylcarnitine | Plasma | 10 | 4.40% | Normal |
| 3 | Lactate | Serum | 10 | 4.40% | Normal |
| 3 | Valine | Serum | 10 | 4.40% | Normal |
| 3 | Leucine | Serum | 10 | 4.40% | Normal |
| 3 | Isoleucine | Serum | 10 | 4.40% | Normal |
| 3 | Phenylalanine | Serum | 10 | 4.40% | Normal |
| 4 | Glutamine | Serum | 8 | 3.50% | Reverse |

SIM (P) stands for Score of individual marker (punctuation)

TABLE 3
Inflammation cluster
Inflammation cluster

| | | | | | Normal/ |
|---|---|---|---|---|---|
| # | Biomarker | Biofluid | SIM (P) | Weight (%) | Reverse |
| 1 | CRP | Serum | 25 | 11.10% | Normal |
| 1 | N-acetylglycoproteins | Serum | 25 | 11.10% | Normal |
| 1 | MCP-1 | Plasma | 25 | 11.10% | Normal |
| 2 | TNFα | Plasma | 23 | 10.20% | Normal |
| 3 | IL-6 | Plasma | 20 | 8.80% | Normal |
| 4 | IL-10 | Plasma | 18 | 8.00% | Normal |
| 5 | SFAs | Serum | 17 | 7.50% | Normal |
| 6 | sICAM1 | Plasma | 15 | 6.60% | Normal |
| 7 | LPCs (total) | Plasma | 13 | 5.80% | Reverse |
| 7 | LBP | Plasma | 13 | 5.80% | Normal |
| 8 | DHA | Serum | 10 | 4.40% | Reverse |
| 8 | sCD14 | Plasma | 10 | 4.40% | Normal |
| 9 | Linoleic acid | Serum | 7 | 3.10% | Normal |
| 10 | PUFAs | Serum | 5 | 2.20% | Reverse |

SIM (P) stands for Score of individual marker (punctuation)

TABLE 4
Oxidative stress cluster
Oxidative stress cluster

| | | | | | Normal/ |
|---|---|---|---|---|---|
| # | Biomarker | Biofluid | SIM (P) | Weight (%) | Reverse |
| 1 | 8-iso-PGF2α | Urine | 25 | 18.12% | Normal |
| 2 | LDLox | Plasma | 21 | 15.22% | Normal |
| 2 | Uric acid | Serum | 21 | 15.22% | Reverse |
| 3 | Allantoin | Urine | 18 | 13.04% | Normal |
| 4 | Betaine | Urine | 15 | 10.87% | Normal |
| 5 | Pseudouridine | Urine | 13 | 9.42% | Normal |
| 6 | Dimethylglycine | Urine | 10 | 7.25% | Normal |
| 6 | Methionine | Serum | 10 | 7.25% | Reverse |
| 7 | Glycine | Serum | 5 | 3.62% | Reverse |

SIM (P) stands for Score of individual marker (punctuation)

TABLE 5
Microbiota metabolism cluster
Microbiota

| | | | | | Normal/ |
|---|---|---|---|---|---|
| # | Biomarker | Biofluid | SIM (P) | Weight (%) | Reverse |
| 1 | TMA | Urine | 25 | 20.16% | Normal |
| 2 | Betaine | Urine | 20 | 16.13% | Normal |
| 2 | Choline | Serum | 20 | 16.13% | Normal |
| 3 | DMA | Urine | 15 | 12.10% | Normal |
| 4 | Dimethylglycine | Urine | 12 | 9.68% | Normal |
| 5 | LBP | Plasma | 10 | 8.06% | Normal |
| 6 | Succinate | Urine | 8 | 6.45% | Reverse |
| 6 | Lactate | Serum | 8 | 6.45% | Normal |
| 7 | Acetate | Urine | 6 | 4.83% | Normal |

SIM (P) stands for Score of individual marker (punctuation)

The methodology was validated with interventional studies with healthy and obese volunteers. These studies have demonstrated the effectivity of the method in the improvement of health status according to the algorithm described before.

The method allowed to identify how to operate with biomarkers under research, supported by a solid body of evidence but lacking consensus or established thresholds to differentiate between health and disease or altered metabolic states. This is a limitation with some markers that implies that an individual cannot be classified in absolute or binary terms of presence or absence of a phenotype. Thus, the strategy applied for classifying in deviating-from-health graduations was applied. This was done by defining the overall distribution of a biomarker in the general studied population to, subsequently, determine whether an individual falls in higher, lower or middle ranges. Since associations between a given biomarker and altered health states are already known, such an approach provided initial information of whether that biomarker pointed to a higher risk of developing a given phenotype.

Data were analysed by Principal Component Analysis (PCA). Representation of each subject according to their respective values in PC1 vs PC2 was done in plots.

The detected distributions in the PCA suggested that each metabolic score showed differential trends throughout the population of study, and therefore that it could capture different metabolic signatures within a population (data not shown).

### Example 2. Validation Cohort and Implementation of Diet Selection

The approach disclosed in Example 1 was used to provide dietary recommendations in a single blind, parallel, randomized, controlled nutritional intervention study registered in clinicaltrials.gov (NCT04641559). Briefly, 193 volunteers were randomly allocated to control or personalized interventions. Control subjects (n=55) received standard dietary recommendations. Volunteers in personalized groups (n=68 and n=70) were allocated to dietary plans targeting carbohydrate metabolism, lipid metabolism, microbiota, inflammation, or oxidative stress according to the higher metabolic score calculated as described above.

Effects of specific dietary plans on all metabolic scores were analysed as Intention To Treat by means of linear mixed models with participants as random effect and fixed effects for time (two levels, baseline and 21 weeks), intervention groups (control vs Carbohydrate, Lipid, inflammation, Oxidative Stress or Microbiota), and their interaction with sex and age as covariate. Intragroup comparisons were conducted per protocol by paired Mann-Whitney or Student's t and by unpaired comparisons when analysed per intention to treat.

Results are shown in table 6 A to G.

TABLE 6A
Control diet
Control scores

| Cluster | Baseline | At 21 weeks |
|---|---|---|
| Lipid cluster, score | −0.32 ± 0.45 | −0.26 ± 0.35 |
| Carbohydrate cluster, score | −0.16 ± 0.46 | 0.01 ± 0.51* |
| Inflammation cluster, score | −0.08 ± 0.33 | −0.07 ± 0.43 |
| Oxidative stress cluster, score | 0.05 ± 0.31 | 0.18 ± 0.46 |
| Microbiota cluster, score | −0.40 ± 0.36 | −0.42 ± 0.38 |

*denotes statistical significance (p-value < 0.05) according to student's t-test or Mann-Whitney U test

TABLE 6 B
Diet for Carbohydrates managing
Test Carbohydrates diet score

| Cluster | Baseline | At 21 weeks |
|---|---|---|
| Lipid cluster, score | −0.12 ± 0.34 | −0.08 ± 0.35 |
| Carbohydrate cluster, score | 0.52 ± 0.4 | 0.29 ± 0.50* |
| Inflammation cluster, score | −0.11 ± 0.38 | −0.06 ± 0.52 |
| Oxidative stress cluster, score | −0.13 ± 0.31 | 0.04 ± 0.36 |
| Microbiota cluster, score | −0.35 ± 0.23 | −0.70 ± 0.19* |

*denotes statistical significance (p-value < 0.05) according to student's t-test or Mann-Whitney U test

TABLE 6C
Diet for Inflammation managing
Test Inflammation diet score

| Cluster | Baseline | At 21 weeks |
|---|---|---|
| Lipid cluster, score | −0.69 ± 0.35 | −0.31 ± 0.31* |
| Carbohydrate cluster, score | −0.53 ± 0.43 | −0.23 ± 0.28* |
| Inflammation cluster, score | 0.58 ± 1.81 | −0.06 ± 0.26 |
| Oxidative stress cluster, score | −0.12 ± 0.32 | 0.20 ± 0.31* |
| Microbiota cluster, score | −0.47 ± 0.33 | −0.53 ± 0.21 |

*denotes statistical significance (p-value < 0.05) according to student's t-test or Mann-Whitney U test

TABLE 6D
Diet for Lipid managing
Test Lipid diet score

| Cluster | Baseline | At 21 weeks |
|---|---|---|
| Lipid cluster, score | −0.03 ± 0.30 | 0.00 ± 0.30 |
| Carbohydrate cluster, score | −0.18 ± 0.38 | −0.03 ± 0.38* |
| Inflammation cluster, score | −0.10 ± 0.29 | −0.04 ± 0.35 |
| Oxidative stress cluster, score | 0.07 ± 0.26 | 0.16 ± 0.42 |
| Microbiota cluster, score | −0.47 ± 0.26 | −0.50 ± 0.22 |

*denotes statistical significance (p-value < 0.05) according to student's t-test or Mann-Whitney U test

TABLE 6E
Diet for Microbiota managing
Test Microbiota diet score

| Cluster | Baseline | At 21 weeks |
|---|---|---|
| Lipid cluster, score | −0.57 ± 0.4 | −0.42 ± 0.42 |
| Carbohydrate cluster, score | −0.23 ± 0.36 | 0.03 ± 0.30* |
| Inflammation cluster, score | 0.09 ± 0.87 | −0.14 ± 0.35 |
| Oxidative stress cluster, score | 0.17 ± 0.37 | 0.33 ± 0.7 |
| Microbiota cluster, score | 0.07 ± 0.44 | −0.38 ± 0.25* |

*denotes statistical significance (p-value < 0.05) according to student's t-test or Mann-Whitney U test

TABLE 6F
Diet for Oxidative stress managing
Test Oxidative stress scores

| Cluster | Baseline | At 21 weeks |
|---|---|---|
| Lipid cluster, score | −0.55 ± 0.27 | −0.58 ± 0.3 |
| Carbohydrate cluster, score | −0.53 ± 0.28 | −0.37 ± 0.19 |
| Inflammation cluster, score | −0.31 ± 0.18 | −0.22 ± 0.26* |
| Oxidative stress cluster, score | 0.32 ± 0.4 | 0.07 ± 0.34* |
| Microbiota cluster, score | −0.45 ± 0.24 | −0.59 ± 0.19* |

*denotes statistical significance (p-value < 0.05) according to student's t-test or Mann-Whitney U test

TABLE 6G
Statistical analysis inter-groups stablishing Control group as the
reference in linear mixed models with subjects as random effects
and time, age and sex as fixed effects. P-values for group and group
× time interaction for each dietary plan versus the control
group (CARB, INFL, LIPID, MB, OXIS) are indicated in plain text.

| Cluster | P group | P time | P group*time |
|---|---|---|---|
| Lipid cluster, score | <0.001** | 0.3** | 0.018** |
| | CARB: 0.018 | | CARB: 0.78 |
| | INFL: <0.001 | | INFL: 0.008 |
| | LIPID: 0.001 | | LIPID: 0.65 |
| | MB: 0.030 | | MB: 0.30 |
| | OXIS: 0.31 | | OXIS: 0.27 |
| Carbohydrate cluster, score | <0.001** | 0.004** | <0.001** |
| | CARB: <0.001 | | CARB: <0.001 |
| | INFL: 0.003 | | INFL: 0.24 |
| | LIPID: 0.73 | | LIPID: 0.83 |
| | MB: 0.65 | | MB: 0.26 |
| | OXIS: <0.001 | | OXIS: 0.82 |
| Inflammation cluster, score | <0.001** | 0.99** | 0.010** |
| | CARB: 0.71 | | CARB: 0.71 |
| | INFL: <0.001 | | INFL: 0.001 |
| | LIPID: 0.76 | | LIPID: 0.77 |
| | MB: 0.24 | | MB: 0.29 |
| | OXIS: 0.10 | | OXIS: 0.57 |
| Oxidative stress cluster, score | <0.001** | 0.072** | <0.001** |
| | CARB: 0.036 | | CARB: 0.73 |
| | INFL: 0.11 | | INFL: 0.24 |
| | LIPID: 0.98 | | LIPID: 0.70 |
| | MB: 0.35 | | MB: 0.76 |
| | OXIS: <0.001 | | OXIS: <0.001 |
| Microbiota cluster, score | <0.001** | 0.55** | <0.001** |
| | CARB: 0.47 | | CARB: 0.001 |
| | INFL: 0.35 | | INFL: 0.74 |
| | LIPID: 0.18 | | LIPID: 0.98 |
| | MB: <0.001 | | MB: <0.001 |
| | OXIS: 0.70 | | OXIS: 0.24 |

Global p-values for group, time and their interaction were calculated by ANOVA on the linear mixed models and are indicated by two consecutive asterisks (**).
CARB: carbohydrate;
INFL: Inflammation;
LIPID: Lipid;
MB: Microbiota:
OXIS: Oxidative stress;
**Global p-values for group, time and their interaction were calculated by ANOVA on the linear mixed models

From the data in the Tables 6A to 6G, the following conclusions apply:

Carbohydrate subgroup presented beneficial effects on adiponectin levels, which were maintained between visit 1 and 2, contrary to the other dietary plans showing decreased levels. A similar result was observed for glutamate. Moreover, this intervention was the only showing an important significant decrease of circulating branched chain amino acids valine, leucine, isoleucine and aromatic amino acids phenylalanine and tyrosine. Altogether, Carbohydrate Score was significantly decreased by the Carbohydrate-targeted dietary plan. Moreover, Microbiota Score was ameliorated, reflecting, at least, the significant decrease of LBP, TMAO and DMA. Other scores (i.e., inflammation, Lipid, Oxidative Stress) paralleled the trends shown by volunteers in the Control group.

Inflammation subgroup showed positive outcomes versus Control in inflammation biomarkers such as increased glycine and decreased DMG, CRP and NAG. These beneficial effects are reflected in the statistically significant amelioration of the Inflammation Score. Nevertheless, these results were accompanied by statistically significant increase of the Lipid Score, clearly reflecting, at least, the significant increase in levels of LDL and decrease of HDL together with changes in other parameters. Anthropometrics were also negatively affected by the Inflammation intervention. Thus, fat mass significantly increased while lean mass was reduced. Analysis of this subgroup indicates that the Scoring system can capture both beneficial and detrimental effects of interventions, and that this particular intervention caused undesired side effects in lipid metabolism. This is not a negative feature of the method at hand, but an indication of the need of follow-up and of the adjustment of certain diets. On the other side, these data regarding the effect on lipid score caused by a diet thought for the “inflammation group”, allow to readjust diets in a subject in terms of combining a diet considering both scores, that of the inflammation cluster and that of the lipid metabolism cluster.

Lipid subgroup showed slight changes with the applied diet. Thus, significant increases in circulating PUFA and MUFA and the maintenance of Oleic acid levels contrasted with increased linoleic, Leptin and SFA. As a result, no differences were detected between the dietary plan targeting lipids and the control group in any of the calculated metabolic Scores.

Microbiota subgroup presented beneficial outcomes versus the control group in markers of microbiota status and related processes such as pseudouridine, glycine, betaine, dimethylglycine, TMA, and DMA. Altogether, these changes resulted in beneficial significant decrease in the Microbiota Score respect the control group.

Oxidative stress subgroup presented positive outcomes versus control volunteers in different biomarkers of Oxidative Stress. Thus, this intervention was the only one that significantly reduced urine Isoprostanes, the gold standard for oxidative stress, together with levels of urine 8-OH-dG. Additionally, some biomarkers of microbiota status were also ameliorated versus the control group, such as DMG, TMAO and DMA. Moreover, this intervention showed a tendency towards decreased blood pressure, both systolic and diastolic, and a slight but significant decrease in body weight and BMI likely due to decreased lean mass and maintained fat mass. This dietary plan was the only one reducing the Oxidative Stress score.

Except for the Lipid-targeted dietary plan, the different interventions successfully decreased their respective metabolic scores. These results were accompanied by beneficial changes in important biomarkers with current clinical interest for the different metabolic processes that each intervention was targeting. Nevertheless, the Inflammation-targeted dietary plan induced a significant increase in Lipid Score as an undesired side effect.

Altogether, results of metabolic scores are aligned with both clinical outcomes and changes in food choices. Therefore, it has been made plausible that metabolic scores proposed by the invention can capture the different outcomes of nutritional interventions.

For completeness, the present description is also disclosed in the following numbered embodiments:

1.—A method for the selection of a diet for a subject based on health data, comprising:
(i) determining in an isolated sample of the subject two or more markers of two or more of the following metabolic clusters:
(a) carbohydrate metabolism;
(b) lipid metabolism;
(c) inflammation;
(d) microbiota metabolism; and
(e) oxidative stress;
(ii) giving a score to each cluster, which score results from a function considering the levels and/or concentration of the two or more markers in the sample;
(iii) comparing the scores obtained for the two or more of clusters (a) to (e) to determine a gradation between them; and
(iv) recommending a diet for the subject considering the said gradation and optionally the individual score value in relation to a reference value for that score.

2.—The method according to embodiment 1, wherein the function to get the score of each cluster in step (ii) comprises giving a value to each of the markers of the cluster, which value results from the concentration of the marker detected in the isolated sample, the concentration optionally calculated as a Z-score, modified by a factor.

3.—The method according to any one of embodiments 1-2, wherein the score of each cluster in step (ii) is calculated according to the following formula (2):

$$Score ( cluster ) = {\sum }_{n = 1}^{Ncluster} ( {weight ( cluster )}_{n} * {biomarker ( cluster )}_{n} ) \qquad ( 2 )$$

wherein
   “biomarker (cluster)” is the level and/or concentration of a particular marker determined in the isolated sample for a particular cluster;
   “weight (cluster)” is a factor selected from a rational from −1 to +1;
   “n” is one specific biomarker of the cluster; and
   “Ncluster” is the total amount of biomarkers for that specific cluster.

4.—The method according to embodiment 3, wherein the factor is calculated according to the following formula (1):

$${weight ( cluster )}_{n} = \frac{{SIM ( cluster )}_{n}}{{\sum }_{n = 1}^{Ncluster} {SIM ( cluster )}_{n}} \qquad ( 1 )$$

wherein
   “weight (cluster)” is the weight or factor selected from a rational from −1 to +1;
   “SIM (cluster)” is the Score of individual marker (punctuation) of a particular marker determined in the isolated sample for a particular cluster;
   “n” is one specific biomarker of the cluster; and
   “Ncluster” is the total amount of biomarkers for that specific cluster.

5.—The method according to embodiment 4, wherein the SIM value for each marker is proportionally related to the SIM values for the other determined markers within the same cluster in a proportion falling within the ranges given in Tables F-J, wherein X is selected from 0.75, 0.50, 0.30, 0.10, or 0.

6.—The method according to any one of embodiments 1-5, wherein:
the markers of the (a) carbohydrate metabolism cluster are selected from glucose, homeostatic model assessment of insulin resistance (HOMA-IR), glutamate, uric acid, leptin, adiponectin, insulin, tyrosine, propionylcarnitine, lactate, valine, leucine, isoleucine, phenylalanine, and glutamine;
the markers of the (b) lipid metabolism cluster are selected from LDL-cholesterol, total cholesterol, total polyunsaturated fatty acids (PUFAs (total)), HDL-cholesterol, saturated fatty acids, triglycerides, total monounsaturated fatty acids (MUFAs (total), total lysophosphatidylcholine (LPC total), linoleic acid, docosahexaenoic acid (DHA), oleic acid, choline, 3-hydroxybutyrate, propionylcarnitine, adiponectin, and leptin;
the markers of the (c) inflammation cluster are selected from C-reactive protein (CRP), N-acetylglycoproteins, Monocyte chemoattractant protein-1 (MCP-1), tumour necrosis factor α (TNFα), interleukin-6 (IL-6), interleukin-10 (IL-10), saturated fatty-acids (SFAs), Soluble intercellular adhesion molecule-1 (sICAM1), total Lysophosphatidylcholine (LPC total), lipopolysaccharide binding protein (LBP), docosahexaenoic acid (DHA), soluble cluster of differentiation 14 (sCD14), linoleic acid, and total polyunsaturated fatty acids (PUFAs (total));
the markers of the (d) microbiota metabolism cluster are selected from trimethylamine (TMA) N-oxide of trimethylamine (TMAO), betaine, choline, dimethylamine (DMA), dimethylglycine, lipopolysaccharide binding protein (LBP), succinate, lactate, and acetate; and
the markers of the (e) oxidative stress cluster are selected from 8-iso-prostaglandin F2α (8-iso-PGF2α), 8-Hydroxyguanosine (8-OHdG), oxidized low density lipoprotein (LDLox), uric acid, allantoin, betaine, pseudouridine, dimethylglycine, methionine, and glycine.

7.—The method according to any one of embodiments 1-6, wherein in step (i) the following markers of the (a) cluster are determined in the isolated sample: glucose, homeostatic model assessment of insulin resistance (HOMA-IR), glutamate, uric acid, leptin, adiponectin, insulin, tyrosine, propionylcarnitine, lactate, valine, leucine, isoleucine, phenylalanine, and glutamine.

8.—The method according to any one of embodiments 1-7, wherein in step (i) the following markers of the (b) cluster are determined in the isolated sample: LDL-cholesterol, total cholesterol, total polyunsaturated fatty acids (PUFAs (total)), HDL-cholesterol, saturated fatty acids, triglycerides, total monounsaturated fatty acids (MUFAs (total)), total Lysophosphatidylcholine (LPC total), linoleic acid, docosahexaenoic acid (DHA), oleic acid, choline, 3-hydroxybutyrate, propionylcarnitine, adiponectin, and leptin.

9.—The method according to any one of embodiments 1-8, wherein in step (i) the following markers of the (c) cluster are determined in the isolated sample: C-reactive protein (CRP), N-acetylglycoproteins, Monocyte chemoattractant protein-1 (MCP-1), tumor necrosis factor α (TNFα), interleukin-6 (IL-6), interleukin-10 (IL-10), saturated fatty-acids (SFAs), Soluble intercellular adhesion molecule-1 (sICAM1), total Lysophosphatidylcholine (LPC total), lipopolysaccharide binding protein (LBP), docosahexaenoic acid (DHA), soluble cluster of differentiation 14 (sCD14), linoleic acid, and total polyunsaturated fatty acids (PUFAs (total)).

10.—The method according to any one of embodiments 1-9, wherein in step (i) the following markers of the (d) cluster are determined in the isolated sample: trimethylamine (TMA) N-oxide of trimethylamine (TMAO), betaine, choline, dimethylamine (DMA), dimethylglycine, lipopolysaccharide binding protein (LBP), succinate, lactate, and acetate.

11.—The method according to any one of embodiments 1-10, wherein in step (i) the following markers of the (e) cluster are determined in the isolated sample: 8-iso-prostaglandin F2α (8-iso-PGF2α), 8-Hydroxyguanosine (8-OHdG), oxidized low density lipoprotein (LDLox), uric acid, allantoin, betaine, pseudouridine, dimethylglycine, methionine and glycine.

12.—The method according to any one of embodiments 1-11, wherein markers of two clusters, or alternatively, of three clusters, or alternatively of four clusters, are determined in step (i).

13.—The method according to any one of embodiments 1-12, wherein markers of the five clusters are determined in step (i).

14. A system for the selection of a diet for a subject based on health data, comprising:
a database that stores data determined in an isolated sample of the subject of the two or more markers of two or more of the following metabolic clusters:
(a) carbohydrate metabolism;
(b) lipid metabolism;
(c) inflammation;
(d) microbiota metabolism; and
(e) oxidative stress;
a memory that stores program instructions, including program instructions that are capable of implementing: (i) a function to get a score of the metabolic clusters from the levels and/or concentrations of the two or more markers in the sample; (ii) a comparison of the scores to determine a gradation between them; and (iii) a diet selection for the subject based on the score of the metabolic clusters and their comparison; and
a processor, coupled to the database and the memory, that when executing the program instructions causes the function to get a score of the metabolic clusters from the levels and/or concentrations of the two or more markers in the sample, causes the comparison of the scores to determine a gradation between them, and causes a diet selection for the subject based on the score of the metabolic clusters and their comparison.

15. The system according to embodiment 14 adapted to perform the method according to any one of embodiments 1-13.

### CITATION LIST

Vernocchi, P., Del Chierico, F., & Putignani, L. (2020). Gut Microbiota Metabolism and Interaction with Food Components. International journal of molecular sciences, 21 (10), 3688. https://doi.org/10.3390/ijms21103688
European Patent EP3529379B1

1. A method for the selection of a diet for a subject based on health data, comprising:
(i) determining in an isolated sample of the subject two or more markers of two or more of the following metabolic clusters:
(a) carbohydrate metabolism;
(b) lipid metabolism;
(c) inflammation;
(d) microbiota metabolism; and
(e) oxidative stress;
wherein:
the markers of the (a) carbohydrate metabolism cluster are selected from glucose, homeostatic model assessment of insulin resistance (HOMA-IR), glutamate, uric acid, leptin, adiponectin, insulin, tyrosine, propionylcarnitine, lactate, valine, leucine, isoleucine, phenylalanine, and glutamine;
the markers of the (b) lipid metabolism cluster are selected from LDL-cholesterol, total cholesterol, total polyunsaturated fatty acids (PUFAs (total), HDL-cholesterol, saturated fatty acids, triglycerides, total monounsaturated fatty acids (MUFAs (total)), total lysophosphatidylcholine (LPC total), linoleic acid, docosahexaenoic acid (DHA), oleic acid, choline, 3-hydroxybutyrate, propionylcarnitine, adiponectin, and leptin;
the markers of the (c) inflammation cluster are selected from C-reactive protein (CRP), N-acetylglycoproteins, Monocyte chemoattractant protein-1 (MCP-1), tumour necrosis factor α (TNFα), interleukin-6 (IL-6), interleukin-10 (IL-10), saturated fatty-acids (SFAs), Soluble intercellular adhesion molecule-1 (sICAM1), total Lysophosphatidylcholine (LPC total), lipopolysaccharide binding protein (LBP), docosahexaenoic acid (DHA), soluble cluster of differentiation 14 (sCD14), linoleic acid, and total polyunsaturated fatty acids (PUFAs (total);
the markers of the (d) microbiota metabolism cluster are selected from trimethylamine (TMA) N-oxide of trimethylamine (TMAO), betaine, choline, dimethylamine (DMA), dimethylglycine, lipopolysaccharide binding protein (LBP), succinate, lactate, and acetate; and
the markers of the (e) oxidative stress cluster are selected from 8-iso-prostaglandin F2α (8-iso-PGF2α), 8-Hydroxyguanosine (8-OHdG), oxidized low density lipoprotein (LDLox), uric acid, allantoin, betaine, pseudouridine, dimethylglycine, methionine, and glycine;
(ii) giving a score to each cluster, which score is calculated according to the following formula (2):
Score(cluster)=Σₙ₌₁^{Ncluster}(weight(cluster)ₙ*biomarker(cluster)ₙ) (2)
wherein
“biomarker (cluster)” is the level and/or concentration of a particular marker determined in the isolated sample for a particular cluster, the concentration optionally calculated as a Z-score;
“n” is one specific biomarker of the cluster;
“Ncluster” is the total amount of biomarkers for that specific cluster; and
“weight (cluster)” is the weight or factor selected from a rational from −1 to +1, which is calculated according to the following formula (1):
$${weight ( cluster )}_{n} = \frac{{SIM ( cluster )}_{n}}{{\sum }_{n = 1}^{Ncluster} {SIM ( cluster )}_{n}} \qquad ( 1 )$$
wherein
“n” and “Ncluster” are as defined above; and
“SIM (cluster)” is the Score of individual marker (punctuation) of a particular marker determined in the isolated sample for a particular cluster; wherein the SIM value for each marker is proportionally related to the SIM values for the other determined markers within the same cluster in a proportion falling within the ranges given in Tables F-J, wherein X is selected from 0.75 to 0;
(iii) comparing the scores obtained for the two or more of clusters (a) to (e) to determine a gradation between them; and
(iv) recommending a diet for the subject considering the said gradation and optionally the individual score value in relation to a reference value for that score.

2. The method according to claim 1, wherein X is selected from 0.50 to 0.

3. The method according to any one of claims 1-2, wherein X is selected from 0.25 to 0.

4. The method according to any one of claims 1-3, wherein X is selected from 0.10 to 0.

5. The method according to any one of claims 1-4, wherein X is 0.

6. The method according to any one of claims 1-5, wherein in step (i) the following markers of the (a) cluster are determined in the isolated sample: glucose, homeostatic model assessment of insulin resistance (HOMA-IR), glutamate, uric acid, leptin, adiponectin, insulin, tyrosine, propionylcarnitine, lactate, valine, leucine, isoleucine, phenylalanine, and glutamine.

7. The method according to any one of claims 1-6, wherein in step (i) the following markers of the (b) cluster are determined in the isolated sample: LDL-cholesterol, total cholesterol, total polyunsaturated fatty acids (PUFAs (total), HDL-cholesterol, saturated fatty acids, triglycerides, total monounsaturated fatty acids (MUFAs (total)), total Lysophosphatidylcholine (LPC total), linoleic acid, docosahexaenoic acid (DHA), oleic acid, choline, 3-hydroxybutyrate, propionylcarnitine, adiponectin, and leptin.

8. The method according to any one of claims 1-7, wherein in step (i) the following markers of the (c) cluster are determined in the isolated sample: C-reactive protein (CRP), N-acetylglycoproteins, Monocyte chemoattractant protein-1 (MCP-1), tumor necrosis factor α (TNFα), interleukin-6 (IL-6), interleukin-10 (IL-10), saturated fatty-acids (SFAs), Soluble intercellular adhesion molecule-1 (sICAM1), total Lysophosphatidylcholine (LPC total), lipopolysaccharide binding protein (LBP), docosahexaenoic acid (DHA), soluble cluster of differentiation 14 (sCD14), linoleic acid, and total polyunsaturated fatty acids (PUFAs (total).

9. The method according to any one of claims 1-8, wherein in step (i) the following markers of the (d) cluster are determined in the isolated sample: trimethylamine (TMA) N-oxide of trimethylamine (TMAO), betaine, choline, dimethylamine (DMA), dimethylglycine, lipopolysaccharide binding protein (LBP), succinate, lactate, and acetate.

10. The method according to any one of claims 1-9, wherein in step (i) the following markers of the (e) cluster are determined in the isolated sample: 8-iso-prostaglandin F2α (8-iso-PGF2α), 8-Hydroxyguanosine (8-OHdG), oxidized low density lipoprotein (LDLox), uric acid, allantoin, betaine, pseudouridine, dimethylglycine, methionine and glycine.

11. The method according to any one of claims 1-10, wherein markers of two clusters, or alternatively, of three clusters, or alternatively of four clusters, are determined in step (i).

12. The method according to any one of claims 1-11, wherein markers of the five clusters are determined in step (i).

13. A computer-implemented method comprising at least one of the following steps (ii), (iii) and (iv) of the method as defined in any one of claims 1-12.

14. The computer-implemented method according to claim 13 comprising steps (ii), (iii) and (iv) of the method as defined in any one of claims 1-12.

15. The computer-implemented method according to any one of claims 13-14 wherein data determined in an isolated sample following step (i) of the method as defined in any one of claims 1-12 is used to perform step (ii).

16. A data processing system comprising means for carrying out steps (ii), (iii) and (iv) of the method of any one of claims 1-12.

17. The system according to claim 16 wherein means comprise:
a database that stores data determined in an isolated sample of the subject of the two or more markers of two or more of the following metabolic clusters:
(a) carbohydrate metabolism;
(b) lipid metabolism;
(c) inflammation;
(d) microbiota metabolism; and
(e) oxidative stress;
a memory that stores program instructions, including program instructions that are capable of implementing: (i) a function to get a score of the metabolic clusters from the levels and/or concentrations of the two or more markers in the sample; (ii) a comparison of the scores to determine a gradation between them; and (iii) a diet selection for the subject based on the score of the metabolic clusters and their comparison; and
a processor, coupled to the database and the memory, that when executing the program instructions causes the function to get a score of the metabolic clusters from the levels and/or concentrations of the two or more markers in the sample, causes the comparison of the scores to determine a gradation between them, and causes a diet selection for the subject based on the score of the metabolic clusters and their comparison.

18. A computer program comprising instructions which, when the program is executed by a computer, cause the computer to carry out the steps (ii), (iii) and (iv) of the method as defined in any one of claims 1-12.

19. A computer-readable data carrier having stored thereon the computer program as defined in claim 18.
